SEQUENCE LISTING (18 sequences)

SEQ ID NO: 1 - DNA, 24 bp, Artificial Sequence
  Synthetic
ctagctagctccggcctgaacgac

SEQ ID NO: 2 - DNA, 27 bp, Artificial Sequence
  Synthetic
gacgacgacaagagcatgaataaccaa

SEQ ID NO: 3 - DNA, 27 bp, Artificial Sequence
  Synthetic
ttggttattcatgctcttgtcgtcgtc

SEQ ID NO: 4 - DNA, 24 bp, Artificial Sequence
  Synthetic
ccgctcgagttacgccatcactgt

SEQ ID NO: 5 - protein, 10 aa, Artificial Sequence
  Synthetic
MNNQRKKARN

SEQ ID NO: 6 - protein, 10 aa, Artificial Sequence
  Synthetic
KRWGTIKKSK

SEQ ID NO: 7 - protein, 10 aa, Artificial Sequence
  Synthetic
ERNRVSTVQQ

SEQ ID NO: 8 - protein, 10 aa, Artificial Sequence
  Synthetic
ARNTPFNMLK

SEQ ID NO: 9 - protein, 10 aa, Artificial Sequence
  Synthetic
KSKAINVLRG

SEQ ID NO: 10 - protein, 10 aa, Artificial Sequence
  Synthetic
RFSLGMLQGR

SEQ ID NO: 11 - protein, 10 aa, Artificial Sequence
  Synthetic
PPTAGILKRW

SEQ ID NO: 12 - protein, 10 aa, Artificial Sequence
  Synthetic
RVSTVQQLTK

SEQ ID NO: 13 - protein, 10 aa, Artificial Sequence
  Synthetic
RGFRKEIGRM

SEQ ID NO: 14 - DNA, 345 bp, Artificial Sequence
  Synthetic
atgaataaccaacgaaaaaaggcgagaaatacgcctttcaatatgctgaaacgcgagagaaaccgcgtgtcgactgtacaacagctgacaaagagattctcacttggaatgctgcagggacgaggaccattaaaactgttcatggccctggtggcgttccttcgtttcctaacaatcccaccaacagcagggatactgaagagatggggaacaattaaaaaatcaaaagccattaatgttttgagagggttcaggaaagagattggaaggatgctgaacatcttgaacaggagacgcagaactgcaggcatgatcattatgctgattccaacagtgatggcgtaa

SEQ ID NO: 15 - protein, 9 aa, Artificial Sequence
  Synthetic
FSLGMLQGR

SEQ ID NO: 16 - protein, 13 aa, Artificial Sequence
  Synthetic
FLTIPPTAGILKR

SEQ ID NO: 17 - DNA, 486 bp, Artificial Sequence
  Synthetic
atgggcagcagccatcatcatcatcatcacagcagcggcctggtgccgcgcggcagccatatggctagctccggcctgaacgacatcttcgaggctcagaaaatcgaatggcacgaaggcggcgatgacgacgacaagagcatgaataaccaacgaaaaaaggcgagaaatacgcctttcaatatgctgaaacgcgagagaaaccgcgtgtcgactgtacaacagctgacaaagagattctcacttggaatgctgcagggacgaggaccattaaaactgttcatggccctggtggcgttccttcgtttcctaacaatcccaccaacagcagggatactgaagagatggggaacaattaaaaaatcaaaagccattaatgttttgagagggttcaggaaagagattggaaggatgctgaacatcttgaacaggagacgcagaactgcaggcatgatcattatgctgattccaacagtgatggcgtaa

SEQ ID NO: 18 - protein, 161 aa, Artificial Sequence
  Synthetic
MGSSHHHHHHSSGLVPRGSHMASSGLNDIFEAQKIEWHEGGDDDDKSMNNQRKKARNTPFNMLKRERNRVSTVQQLTKRFSLGMLQGRGPLKLFMALVAFLRFLTIPPTAGILKRWGTIKKSKAINVLRGFRKEIGRMLNILNRRRRTAGMIIMLIPTVMA

(19) United States
(12) Patent Application Publication
(10) Pub. No.: US 2015/0291664 A1
(43) Pub. Date: Oct. 15, 2015

(54) BIOTINYLATED PROTEIN

(71) Applicants: Mah Lee Mary NG, Singapore (SG); Krupakar PARTHASARATHY, Singapore (SG); Mun Keat CHONG, Singapore (SG); NATIONAL UNIVERSITY OF SINGAPORE, Singapore (SG)

(72) Inventors: Mah Lee Mary Ng, Singapore (SG); Krupakar Parthasarathy, Singapore (SG); Mun Keat Chong, Singapore (SG)

(73) Assignee: National University of Singapore, Singapore (SG)

(21) Appl. No.: 14/646,845

(22) PCT Filed: Nov. 22, 2013
(86) PCT No.: PCT/SG2013/000501

§ 371 Date: May 22, 2015

(30) Foreign Application Priority Data
Nov. 22, 2012 (SG) 201208602-1

(51) Int. Cl. C07K 14/005; C12N 7/00; A61K 39/12

(52) U.S. Cl. CPC C07K 14/005; A61K 39/12; C12N 7/00; C07K 2319/02; C12N 2770/24134; C12N 2770/24122; A61K 38/00

(57) ABSTRACT

The present invention relates to a fused protein. In particular, it relates to biotinylated protein comprising a protein selected from a group consisting of flavivirus structural and non-structural (NS) proteins.

### CROSS-REFERENCE TO RELATED APPLICATIONS

This application is a U.S. National Phase of International Patent Application No. PCT/SG2013/000501, filed Nov. 22, 2013, which claims the benefit of Singapore Application No. 201208602-1, filed Nov. 22, 2012, each of which is incorporated by reference in its entirety herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 5,940 Byte ASCII (Text) file named “720840_ST25.TXT,” dated Jun. 4, 2015.

### FIELD OF THE INVENTION

The present invention relates to a biotinylated protein. In particular, it relates to biotinylated flavivirus structural and non-structural proteins.

### BACKGROUND OF THE INVENTION

The family Flaviviridae contains at least 70 arthropod-transmitted viruses, many of which infect humans and other vertebrates. Dengue virus (DENV) belongs to the Flavivirus genus within the Flaviviridae family. Other members of the Flavivirus genus include Yellow Fever virus (YFV), West Nile virus (WNV), Kunjin virus (KUNV), Japanese Encephalitis virus (JEV), and Tick-Borne Encephalitis virus (TBEV), just to name a few. All flaviviruses, including West Nile Virus, St Louis encephalitis, dengue, Japanese encephalitis, yellow fever and Kunjin viruses share similar size, symmetry and appearance. Despite the fact that flaviviruses may use different process to enter a host cell, such as endocytosis (described for West Nile Virus and Kunjin Virus) and direct fusion of the cell (described for dengue and Encephalitis Virus), entry of all flaviviruses into the host-cell involves an interaction between the virus and a receptor of the cell.

DENV infection encompasses a wide spectrum of severity ranging from mild asymptomatic Dengue Fever (DF) to critical and fatal Dengue Haemorrhagic Fever (DHF)/Dengue Shock Syndrome (DSS). DENV causes about 15,000 deaths annually and it is estimated that more than 2.5 billion people are at risk of DENV infection in more than 100 countries (Gubler, 2002). However, anti-viral drug and vaccine are yet to be available in the market.

The whole mature virion particle is about 50 nm in diameter. Each virion contains a single positive-stranded RNA that is encapsulated by multiple copies of capsid (C) protein to form a spherical cage-like structure called nucleocapsid or core. The core is approximately 30 nm in diameter which can be seen as a dense particle under electron microscope (Hase et al., 1987). Nucleocapsid is surrounded by a 10 nm thick lipid bilayer derived from host cell membrane anchored with 180 membrane (M) protein and 180 envelope (E) protein (Zhang et al., 2003).

DENV C protein is the first structural protein found in the open reading frame (ORF) of its genome. It is one of the three structural proteins that form a mature virus. Although the sequence homology of C proteins among other flaviviruses is poorly conserved (FIGS. 1A and 1B), they are still structurally and functionally similar. The C-terminal hydrophobic signal sequence of full-length DENV C protein is cleaved by its NS2B-NS3 proteins to generate mature DENV C protein (Amberg and Rice, 1999, Markoff, 1989). DENV C protein has a molecular weight of about 12-15 kDa and is a highly basic protein that contains about 25% of lysine and arginine residues. The high basic-residue content confers its RNA binding property to neutralize the negatively-charged viral RNA. This explains the main function of C protein which is to encapsidate its viral RNA and form nucleocapsid.

Other than its structural function, DENV C protein is also known to carry non-structural functions. It is currently believed that multifunctional C protein is essential in the viral replication, assembly, RNA encapsidation, as well as pathogenesis. The role of C protein in inducing apoptosis by activating caspase-3 and caspase-9 leading to mitochondrial dysfunction was reported (Yang et al., 2008). Recently, we discovered that WNV and DENV C protein interact with human Sec3 exocyst protein (hSec3p) to antagonize the antiviral effect caused by hSec3p (Bhuvanakantham et al., 2010). Moreover, DENV C protein was also known to localize in the nucleus of infected cells during replication (Tadano et al., 1989). We found that C protein was transported into the nucleus by importain-α/β complex and the loss of the nuclear translocation ability affected virus replication (Bhuvanakantham et al., 2009). However, the exact role of C protein in the nucleus still remains unclear and further investigation is warrant.

There is also a lack of information about the interaction of DENV C protein with either prM and E proteins or even its RNA genome. The binding of NS2B-NS3 protein with C protein and how it cleaves C-prM polyprotein is also unclear. This is mainly due to the difficulty of expressing and purifying full-length C protein for functional and structural studies. The NMR structure of DENV C protein studied thus far only encompasses 80 residues from 21st to 100th amino acid residues (Ma et al., 2004). The N-terminus is removed from the structural study because it is conformationally labile and unstable (Jones et al., 2003). The hydrophobic C-terminus of DENV C protein is also excluded due to its solubility problem. Nevertheless, the N-terminus of C protein is found to be antigenic as predicted bioinformatically (FIG. 1C). This is also supported by Puttikhunt and co-workers (2009) that the N-terminus of DENV C protein was immunogenic in mice. Recently, Samsa and colleagues (2012) found that the first 18 amino acid residues from the N-terminus were essential for virus particle assembly. Taken together, these suggest that the N-terminus of DENV C protein has important roles in the pathogenesis and viral replication.

Biotinylation is a popular process in protein engineering to ease detection and purification (Chapman-Smith and Cronan, 1999, Cull and Schatz, 2000). Biotinylated hemagglutinin, for instance, was generated to develop a subtype-specific serological assay to diagnose influenza A virus in patients' sera (Postel, Letzel, Muller, Ehricht, Pourquier, Dauber, Grund, Beer and Harder, 2011). One of the advantages of using biotinylated protein is that the detection sensitivity of the biotinylated protein is greatly enhanced by the high affinity and specificity between biotin and streptavidin (Bayer and Wilchek, 1990). This advantage is exploited for techniques such as co-immunoprecipitation or library screening of interaction proteome (He et al., 2009, Markham et al., 2007, Moreland et al., 2010). Not only that, site-specific biotinylation was also developed as a means for molecular labeling and imaging (Howarth and Ting, 2008, Sueda et al., 2011).

There are various approaches to biotinylate a protein, either chemically or enzymatically (Cull and Schatz, 2000). Various commercial kits are available in the market for chemical biotinylation by conjugating biotin molecules on proteins or antibodies that contain primary amines. However, chemical biotinylation may lead to non-specific and non-homogenous incorporation of biotin which might result in possible loss of activity of the protein (Bayer and Wilchek, 1990). Moreover, another step of removing the excess biotin reagent will increase the chance of losing more proteins.

### SUMMARY OF THE INVENTION

The challenge of studying this protein is to generate pure non-truncated, full-length C proteins for structural and functional studies. This is mainly due to its small molecular weight, highly positively-charged, stability and solubility properties. Full-length DENV C protein was expressed and purified for structural and functional studies. Since biotinylation has been widely used to date for purification, detection, diagnostic, protein-protein interaction studies, imaging studies, and other molecular studies (Chapman-Smith and Cronan, 1999, de Boer et al., 2003, Howarth and Ting, 2008, Postel et al., 2011, Qi and Katagiri, 2011), a biotinylation site was engineered into C protein to ease further downstream experiments. Using expression and purification strategies, the present inventors have successfully obtained functional biotinylated full-length DENV C protein.

In particular, the present inventors have developed a strategy to construct, express, biotinylate and purify non-truncated, full-length DENV C protein. A 6×His tag and a biotin acceptor peptide (BAP) were cloned at the N-terminus of C protein using overlapping extension-PCR (OE-PCR) method for site-specific biotinylation. The final construct was inserted into pET28a plasmid and BL-21 (CodonPlus) expression competent cell strain was selected as there are 12% rare codons in the C protein sequence. Strikingly, and advantageously, the inventors found that the recombinant proteins with BAP were biotinylated endogenously with high efficiency in Escherichia coli BL-21 strains. To purify this his-tagged C protein, nickel-nitriloacetic acid (Ni-NTA) affinity chromatography was first carried out under denaturing condition. After stepwise dialysis and concurrent refolding, ion exchange-fast protein liquid chromatography (IEX-FPLC) was performed to further separate the residual contaminants. To obtain C protein with high purity, final round of purification with size exclusion chromatography was carried out and a single peak corresponding to C protein was attained. With this optimized sequential purification protocol, the inventors successfully generated pure biotinylated full-length DENV C protein. The functionality of this purified non-truncated DENV C protein was examined and it was found to be suitable for structural and molecular studies.

Advantageously, biotinylated DENV C protein is useful for various cellular, molecular and imaging experiments because of the high detection limit while the function of the protein is still well preserved.

Still advantageously, the fused protein may be potentially used as a candidate protein for screening inhibitors for virus assembly. Also, the fused protein may be used for screening aptamers (DNA, RNA, Peptide and modified aptamer) which can be used in diagnostics or therapeutics. In particular, it may be used for screening specific blockers for flavivirus entry and assembly. In addition, it may also be used for raising antibodies in mice/rabbit models which might have potential application in diagnostics and therapeutics.

In a first aspect of the invention, there is provided a fused protein comprising a moiety and a protein selected from a group consisting of flavivirus structural and non-structural (NS) proteins. Preferably, the flavivirus structural protein is a flavivirus capsid protein or a flavivirus envelope protein.

By “fused protein”, we include any protein that is produced and expressed through the joining of two of more genes which originally coded for separate proteins. It then follows that the translation of this fused gene results in a single polypeptide with functional properties derived from each of the original proteins.

Preferably, the moiety is a readily detectable moiety.

By a “readily detectable moiety”, we include the meaning that the moiety is one which, when located at the target site, for example to the target protein, may be allowed for the detection of that protein. Thus, the compounds of this embodiment of the present invention may be useful in detection and diagnosis. Preferably, the moiety is a biotin acceptor signal peptide.

Preferably, the flavivirus is a Dengue virus or a West Nile virus. Still preferably, the biotin acceptor peptide is fused with a West Nile envelope Domain III protein. Alternatively, the biotin acceptor peptide is fused at the N-terminus of the capsid protein.

Preferably, the fused protein is full-length and non-truncated.

In a second aspect of the invention, there is provided a fused protein according to the first aspect of the invention for use in medicine. Typically, the fused protein may be packaged and presented as a medicament or as a diagnostic agent for use in a patient.

In a third aspect of the invention, there is provided an isolated nucleic acid encoding the fused protein according to the first aspect of the invention.

Suitable nucleic acid molecules may readily be synthesised or constructed by the person skilled in the art using routine methods such as those described in Sambrook et al (1989) Molecular cloning, a laboratory manual, Cold Spring Harbor Press, Cold Spring Harbor, N.Y. Typically the nucleic acid is DNA, but it may be RNA. In the following, where DNA is used, unless the context indicates to the contrary, this also includes RNA.

The nucleic acid is then expressed in a suitable host to produce a polypeptide comprising the compound of this aspect of the invention. Thus, the nucleic acid encoding the polypeptide constituting the fused protein of the invention may be used in accordance with known techniques, appropriately modified in view of the teachings contained herein, to construct an expression vector, which is then used to transform an appropriate host cell for the expression and production of the polypeptide of the invention.

As such, in a fourth aspect of the invention, there is provided an expression vector comprising the nucleic acid according to the second aspect of the invention. Preferably, the expression vector is a plasmid.

Such techniques include those disclosed in U.S. Pat. No. 4,440,859 issued 3 Apr. 1984 to Rutter et al, U.S. Pat. No. 4,530,901 issued 23 Jul. 1985 to Weissman, U.S. Pat. No. 4,582,800 issued 15 Apr. 1986 to Growl, U.S. Pat. No. 4,677,063 issued 30 Jun. 1987 to Mark et al, U.S. Pat. No. 4,678,751 issued 7 Jul. 1987 to Goeddel, U.S. Pat. No. 4,704,362 issued 3 Nov. 1987 to Itakura et al, U.S. Pat. No. 4,710,463 issued 1 Dec. 1987 to Murray, U.S. Pat. No. 4,757,006 issued 12 Jul. 1988 to Toole, Jr. et al, U.S. Pat. No. 4,766,075 issued 23 Aug. 1988 to Goeddel et al and U.S. Pat. No. 4,810,648 issued 7 Mar. 1989 to Stalker, all of which are incorporated herein by reference.

The nucleic acid encoding the polypeptide constituting the fused protein of the invention may be joined to a wide variety of other nucleic acid sequences for introduction into an appropriate host. The companion nucleic acid will depend upon the nature of the host, the manner of the introduction of the nucleic acid into the host, and whether episomal maintenance or integration is desired.

Generally, the nucleic acid is inserted into an expression vector, such as a plasmid, in proper orientation and correct reading frame for expression. If necessary, the nucleic acid may be linked to the appropriate transcriptional and translational regulatory control nucleotide sequences recognised by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a nucleic acid sequence, with any necessary control elements, that codes for a selectable trait in the transformed cell, such as antibiotic resistance. Alternatively, the gene for such selectable trait can be on another vector, which is used to co-transform the desired host cell.

In a fifth aspect of the invention, there is provided a host cell comprising the expression vector according to the fourth aspect of the invention.

Host cells that have been transformed by the recombinant nucleic acid of the invention are then cultured for a sufficient time and under appropriate conditions known to those skilled in the art in view of the teachings disclosed herein to permit the expression of the polypeptide, which can then be recovered.

Many expression systems are known, including bacteria (for example E. coli and Bacillus subtilis), yeasts (for example Saccharomyces cerevisiae), filamentous fungi (for example Aspergillus), plant cells, animal cells and insect cells.

The vectors include a prokaryotic replicon, such as the ColE1 ori, for propagation in a prokaryote, even if the vector is to be used for expression in other, non-prokaryotic, cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a nucleic acid sequence that permits binding of RNA polymerase and transcription to occur. Promoter sequences compatible with exemplary bacterial hosts are typically provided in plasmid vectors containing convenient restriction sites for insertion of a nucleic acid segment of the present invention.

Typical prokaryotic vector plasmids are pUC18, pUC19, pBR322 and pBR329 available from Biorad Laboratories, (Richmond, Calif., USA) and pTrc99A and pKK223-3 available from Pharmacia, Piscataway, N.J., USA.

A typical mammalian cell vector plasmid is pSVL available from Pharmacia, Piscataway, N.J., USA. This vector uses the SV40 late promoter to drive expression of cloned genes, the highest level of expression being found in T antigen-producing cells, such as COS-1 cells.

An example of an inducible mammalian expression vector is pMSG, also available from Pharmacia. This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (Yips) and incorporate the yeast selectable markers his3, trp1, leu2 and ura3. Plasmids pRS413-416 are Yeast Centromere plasmids (YCps).

A variety of methods have been developed to operatively link nucleic acid to vectors via complementary cohesive termini. For instance, complementary homopolymer tracts can be added to the nucleic acid segment to be inserted to the vector nucleic acid. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion as described earlier, is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyse the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of sources including International Biotechnologies Inc, New Haven, Conn., USA.

A desirable way to modify the DNA encoding the polypeptide of this aspect of the invention is to use the polymerase chain reaction as disclosed by Saiki et al (1988) Science 239, 487-491.

In this method the DNA to be enzymatically amplified is flanked by two specific oligonucleotide primers which themselves become incorporated into the amplified DNA. The said specific primers may contain restriction endonuclease recognition sites which can be used for cloning into expression vectors using methods known in the art.

In a sixth aspect of the invention, there is provided an immunogenic composition or vaccine comprising a fused protein according to the first aspect of the invention.

The vaccine may advantageously contain other components, such as adjuvant, attenuated flavivirus, killed flavivirus or subunits thereof, and/or other immunogenic molecules against the flavivirus, in particular against the envelope protein of the flavivirus. The manufacturing process and components to be included in the vaccine are known as such to the person skilled in the art.

The vaccine disclosed herein may be administered by any suitable route, which delivers an immunoprotective amount of the fused protein and other immunogenic components of the vaccine to the patient. Routes of administration of the vaccine, such as, for example, parenteral route, intramuscular route or deep subcutaneous route, are identifiable by a person skilled in the art. Other modes of administration may also be employed, where desired, such as oral administration or via other parenteral routes, i.e., intradermally, intranasally, or intravenously.

A person skilled in the art can determine the appropriate immunoprotective and non-toxic dose of such vaccine to be administered. The appropriate immunoprotective and non-toxic amount of the active agents in the vaccine are in the range of the effective amounts of antigen in conventional vaccines including active agents. The specific dose level for a specific patient will be determined with reference to the age, sex, and general health of the patient. Also, the synergistic effect with other drugs administered as well as the diet of the patient, the time and route of administration, and the degree of protection to be sought, will be taken in consideration to determine the appropriate immunoprotective dose for the patient. The administration can be repeated at suitable intervals, if necessary.

In a seventh aspect of the invention, there is provided a pharmaceutical composition for treating a flavivirus infection in a patient, the composition comprising a therapeutic effective amount of a fused protein or fragment thereof according to the first aspect of the invention and at least one pharmaceutically acceptable carrier, excipient or diluent.

The carrier(s) must be “acceptable” in the sense of being compatible with the fused protein of the invention and not deleterious to the recipients thereof. Typically, the carriers will be water or saline which will be sterile and pyrogen free; however, other acceptable carriers may be used.

The pharmaceutical compositions or formulations of the present invention may be for parenteral administration, for example for intravenous administration. In which case, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Alternatively, or suitably, the pharmaceutical compositions or formulations of the present invention may be for oral administration.

The exact amount required for the treatment of a patient, and route of administration of such amount will vary from patient to patient, depending on the species, age, and general condition of the individual patient, the severity of the infection, the particular antiviral agent and its mode of administration, etc.

In an eighth aspect of the invention, there is provided a method for treating a flavivirus infection in a patient, the method comprising the step of administering to the patient a therapeutically effective amount of a fused protein or fragment thereof according to the first aspect of the invention.

In a ninth aspect of the invention, there is provided a fused protein according to the first aspect of the invention, a pharmaceutical composition according to the seventh aspect of the invention, a nucleic acid according to the third aspect of the invention, or an expression vector according to the fourth aspect of the invention for use in the treatment or prevention of a flavivirus infection in a patient.

In a tenth aspect of the invention, there is provided a use of a fused protein according to the first aspect of the invention, a pharmaceutical composition according to the seventh aspect of the invention, a nucleic acid according to the third aspect of the invention, or an expression vector according to the fourth aspect of the invention in the preparation of a medicament for the treatment or prevention of a flavivirus infection in a patient.

In a eleventh aspect of the invention, there is provided a method for producing a fused protein, the method comprising: (a). obtaining a gene clone derived from a flavivirus capsid protein; (b). obtaining a biotin acceptor peptide gene; (c). producing an expression vector through the joining of the gene clone derived from the flavivirus capsid protein and the biotin acceptor peptide gene; (d). expressing the gene of the fused protein by transforming the expression vector to a host; and (e). purifying the fused protein.

Preferably, the host is Escherichia coli.

Preferably, the purifying step includes the use of urea, non-ionic detergent, ion exchange chromatography and size exclusion chromatography.

In a twelfth aspect of the invention, there is provided a fused protein produced by the method according to the eleventh aspect of the invention for use in the treatment of a flavivirus infection in a patient.

In a thirteenth aspect of the invention, there is provided a method of screening a library of molecules to identify or select one or more molecules thereof which selective bind to a fused protein, or fragment thereof, according to a first aspect of the invention, the method comprising: (a) contacting the library of molecules with the fused protein; and (b) detecting binding of one or more molecules to the fused protein.

Preferably, the method further comprises labelling the molecule with a biotin binding agent and binding is detected by detecting the labelled molecule.

Preferably, the molecule is selected from a group consisting of antibodies and aptamers.

By “antibody”, we include polyclonal or monoclonal antibody unless differently specified. The relevant preparation, is identifiable by a person skilled in the art upon reading of the present disclosure. In the specific examples given, murine polyclonal antibodies were used. Monoclonal antibodies may be obtained by any technique that provides for the production of antibody molecules by continuous cell line culture. These techniques are well known and routinely used in academic and industrial settings. Some techniques include but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl Acad. Sci. USA 80:2026-2030), and the EBV-hybridoma technique (Cole et al., 1985, Monoclonal Antibodies And Cancer Therapy, Alen R. Liss, Inc., pp. 77-96). Antibody fragments, which retain the ability to recognize the antigen of interest, are included as well.

The antibodies are produced using techniques known to those skilled in the art and disclosed, for example, in immunization techniques in vivo or in vitro. These techniques are well known and routinely used in academic and industrial settings.

By “aptamer”, we include any oligonucleic acid or peptide molecules that specifically target and bind to any molecule.

In a fifteenth aspect of the invention, there is provided a diagnostic kit comprising a fused protein according to the first aspect of the invention and instructions for use of the same. Instructions, for example written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

Methods, kits, vaccine and pharmaceutical compositions disclosed herein are particularly used when the flavivirus is a member of the Japanese encephalitis serocomplex, preferably West Nile Virus, Japanese Encephalitis virus, West Valley or a virus such as Dengue and Kunjin virus. Preferably, the vertebrate is a mammal, and, in particular, a human being.

A person skilled in the art can identify modalities, dosages, timing of administration of the methods herein disclosed as well as vehicle carrier auxiliary agents, relative concentration, formulation and modalities of administration of the compositions herein disclosed.

The invention will now be described with reference to the following none limiting figures and examples.

All references herein mentioned are hereby incorporated by reference.

### BRIEF DESCRIPTION OF THE SEVERAL VIEWS OF THE DRAWINGS

FIG. 1. Bioinformatics analysis of flavivirus C protein. (A) Multiple sequence alignment of mosquito-borne flavivirus C protein sequences using ClustalW method in MegAlign, DNASTAR Lasergene 7.2 software. Sequences with the highest consensus strength among the nine flavivirus capsids are highlighted. (B) Sequence distances analysis using ClustalV method shows that the protein sequence similarity among the nine capsids are mostly lower than 50%. (C) Antigenic plot of DENV2 C protein generated by Protean, DNASTAR Lasergene 7.2 software, using Jameson-Wolf methodology. The higher the antigenic index, the more likely the region will be recognized by immune system. The secondary structures are also shown in parallel with the antigenic plot. Alpha helixes are shown in rounded rectangles while beta sheets are shown in normal rectangles. The high antigenicity of the first 20 amino acids are highlighted in dotted box. (DENV1-4—Dengue virus serotype 1-4; JEV—Japanese Encephalitis virus; MVEV—Murray Valley encephalitis virus; UV—Usutu virus; WNV—West Nile virus; YFV—Yellow Fever virus; Accession number of the protein sequence is shown in the bracket.)

FIG. 2. Cloning strategies. (A) Secondary structure prediction of biotinylated C (BNC) and unbiotinylated C (UBNC) proteins show that the BAP will not affect the overall secondary structure formation when it is added to the N-terminal of C protein. Alpha helixes are shown in gray, rounded rectangles while beta sheets are shown in black, normal rectangles. (B) Schematic diagram showing the overlap extension PCR (OE-PCR) technique. Fragment A is designed such that its 3′ overhang is complementary to the 5′ overhang of Fragment B. As such, primer B and primer C are complementary to each other. Both fragments are joined together with the complementary sequence and primers A and D. (C) Plasmid map of pET28aDENVBioCap construct. (D) Final construct of recombinant protein generated. 6×His tag is at the N-terminal followed by thrombin cleavage site and biotin acceptor peptide (BAP). Enterokinase cleavage site is included at the downstream of BAP. 6×His tag is used for affinity purification while BAP is the signal peptide for biotinylation.

FIG. 3. Expression screening of bacterial clones with recombinant DENV C protein and detection of biotinylation. (A) Two expression competent cells are tested, namely BL-21 (DE3) and BL-21-CodonPlus. The presence of C protein band in the IPTG-induced bacterial cell lysate, as indicated by arrow, is detected via SDS-PAGE stained with Coomassie blue (i) and Western blot (ii) using anti-His antibody. The expression of C protein is much higher in CodonPlus competent cells as the band intensity is much higher than normal BL-21 (DE) competent cells. (B) (i) The presence of His tag was detected using monoclonal mouse anti-His antibody and goat anti-mouse horseradish peroxidase (HRP)-conjugated secondary antibody. Bands can be observed in both DENV C proteins with and without biotin acceptor peptide (BAP). The molecular weight of C protein with BAP is higher than C protein without BAP. (ii) When Western-blot is performed using streptavidin-HRP secondary antibody, band is only observed in the DENV C and WNV DIII proteins lanes with BAP. Likewise, band is only observed in the biotinylated (BN) maltose-binding protein (MBP) and not unbiotinylated (UBN) MBP.

FIG. 4. Purification of biotinylated DENV C protein. IPTG-induced bacteria are lyzed in lysis buffer containing 8 M urea and the lysate (L) is incubated with nickel-charged resin overnight. Next, the resin is packed in a column and the flow through (FT) is kept for SDS-PAGE. 20 mM imidazole is used to wash away the unbound proteins (W) and 500 mM imidazole is used to elute the C protein in ten fractions (E1-10). SDS-PAGE is carried out and stained with Coomassie blue (A). Fraction E2 shows the most intense band but there are also many contaminants in the eluates. Western blot is also performed for the samples using anti-His antibody (B) and streptavidin-HRP secondary antibody (C). C protein-corresponding bands can be observed from fraction E2 until E10. The band intensity is the highest in fraction E2 and decreases until fraction E10. A faint C protein dimer-corresponding band can also be observed in fraction E2 as detected by anti-His antibody.

FIG. 5. Sequential purification of DENV C protein using fast protein liquid chromatography (FPLC) system after first round of affinity his-tag purification. Dialysed and refolded DENV C protein from eluates E2 to E10 are injected into Resource MonoQ ion exchange chromatography column. Bound proteins are eluted out using increasing concentration of sodium chloride until final NaCl concentration of 1 M. One high peak can be observed when the sodium chloride concentration reaches 350 mM [A(i)]. The identity of the peak is confirmed to be C protein as detected by ELISA using streptavidin-HRP antibody [A(ii)]. The eluates from the peak are combined and inserted into size exclusion chromatography for further purification. One high peak is observed in the elution profile [B(i)] and this peak is identified to be C protein as confirmed by ELISA using streptavidin-HRP antibody [B(ii)].

FIG. 6. Biotinylation efficiency and functional analysis of purified full-length DENV C protein. (A) (i) ELISA screening of 30 ng biotinylated (BN) and unbiotinylated (UBN) proteins. All three BN maltose-binding protein (MBP), DENV C and WNV DIII proteins show high absorbance at 450 nm as compared to the protein without biotin acceptor peptide (BAP). (ii) Biotin-streptavidin binding assay also shows that only BN proteins bind to streptavidin-magnetic beads and are detected in the eluates. (B) The functionality of DENV C protein is examined via binding assay with Sec3 protein which is known to interact with C protein in host cells. Pure Sec3 protein is coated onto ELISA plate and purified full-length DENV C protein is added into the well for binding. Bovine serum albumin (BSA) is used as negative control. Significant absorbance is detected using streptavidin-horseradish peroxidase antibody in the wells with Sec3 protein coated (Sec3-DENVC) but not with BSA (BSA-DENVC). *p-value<0.05

FIG. 7. FPLC-Gel Filteration Chromatography spectrum corresponding to the West nile virus envelope protein domain III (WNV DIII) and Biotinylated West nile virus envelope protein domain III (BN-WNV DIII).

FIG. 8. ELISA screening for unbiotinylated and Biotinylated proteins. ELISA data for the control proteins (Unbiotinylated and Biotinylated Maltose Binding protein), Unbiotinylated Dengue Virus2 EDIII, Dengue virus3 EDIII, West Nile Virus Envelope DIII, biotinylated (BN) West Nile Virus Envelope DIII (Batch 1 and 2).

FIG. 9. Stepwise representation for the production of biotinylated Dengue virus Capsid protein and West Nile virus Envelope protein DIII.

FIG. 10. Cellular distribution of potential interacting partners of DENV C protein. Total proteins in each cellular compartment are calculated and charted. The diagrams on the top illustrate the cellular localization of each individual protein and some of them may localize in more than one cellular compartment. Majority of the DENV C protein-interacting partners localize in the nucleus followed by cytoplasm. Six proteins are related to cytoskeleton while five are found in the nucleolus and plasma membrane.

FIG. 11. Categorization of the interacting partners of DENV C protein. The function of each interacting protein is analysed and classified. Cell cycle and regulation of transcription and translation are the two major categories of DENV C protein-interacting partners.

FIG. 12 is a graph showing that there were 578 potential DENV C protein-binding partners detected to have z-score value above 1.

FIG. 13 is a graph showing the verification of the binding between potential interaction partners and Dengue virus capsid protein via ELISA.

FIG. 14 is a graph showing the epitope score of nine 10-residue fragments.

FIG. 15 is a graph showing that purified full-length recombinant DENV C protein is recognized by monoclonal antibodies against DENV C peptides.

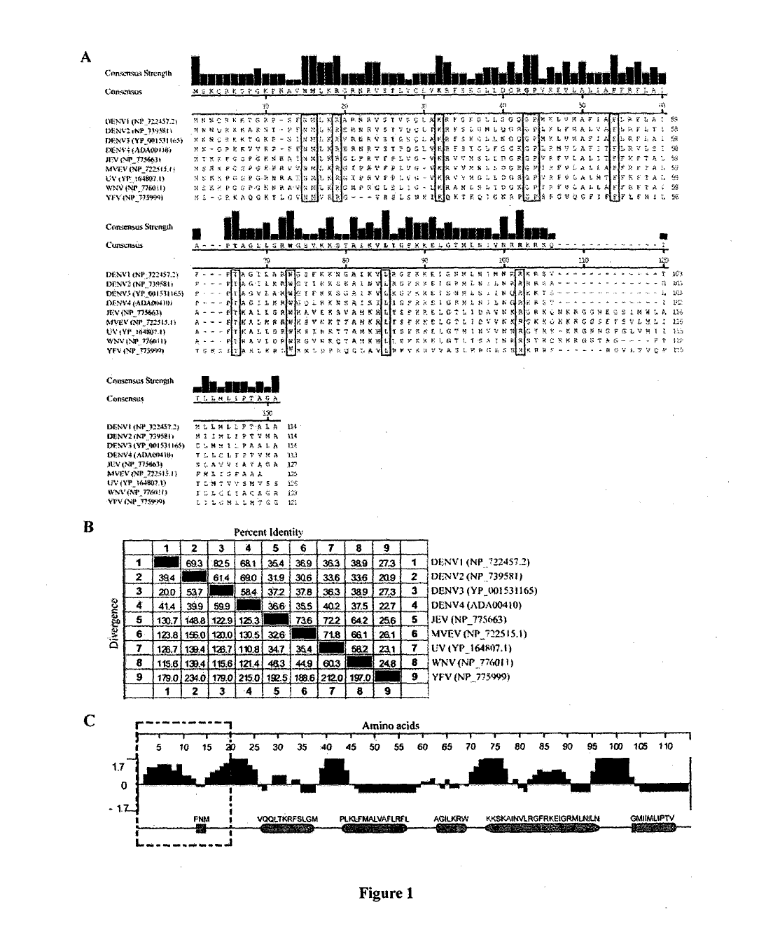

Supplementary FIG. 1. Cation exchange purification of DENV C protein using Resource MonoS column. (A) Dialysed and refolded DENV C proteins after affinity chromatography are injected into Resource MonoS ion exchange chromatography column. Bound proteins are eluted out using increasing concentration of sodium chloride until final concentration of 1 M. However, there is no obvious peak during the elution steps except a small increase of UV absorbance when the concentration of NaCl reaches 40 mM (40%). (B) The presence of C protein is detected by ELISA using streptavidin-HRP antibody. An increasing amount of biotinylated DENV C protein is detected during elution and it reaches plateau at approximately 70% of NaCl (700 mM NaCl).

Supplementary Table 1. DNA (SEQ ID NO: 17) and protein (SEQ ID NO: 18) sequences of full-length DENV C protein

Supplementary Table 2. Rare codon analysis of full-length DENV C protein. Rare codons are in BOLD and underlined. The full-length DENV C DNA sequence is SEQ ID NO: 14.

Supplementary Table 3. Competent cells identified through BLAST analysis against BirA gene.

Supplementary Table 4. MALDI-TOF mass spectrometry analysis of purified DENV C protein, including FSLGMLQGR (SEQ ID NO: 15) and FLTIPPTAGILKR (SEQ ID NO: 16).

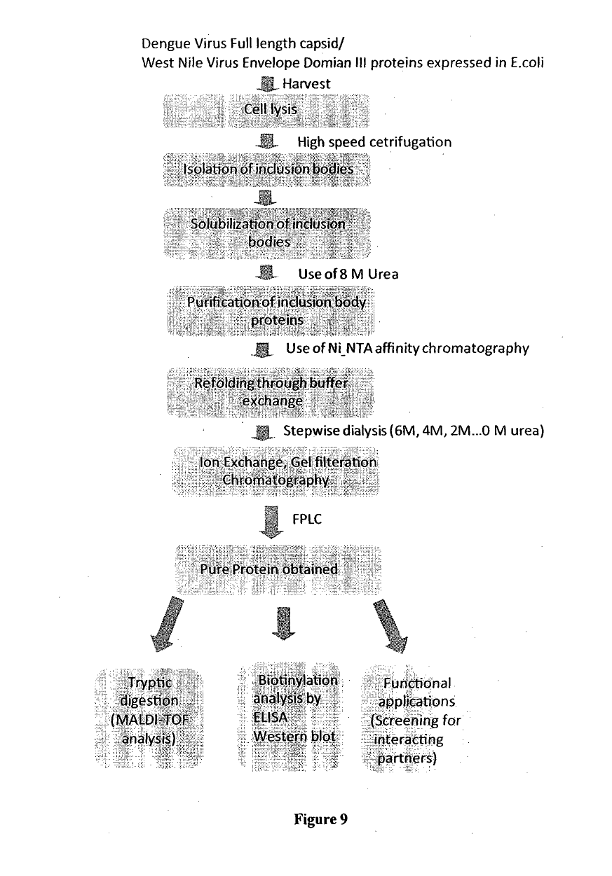

The present invention relates to a fused protein (in vivo site specific biotinylation of a viral (of the Flavivirus genus) protein), a method of producing the protein and application of the protein. The general steps for the production of fused protein—in an embodiment of the present invention, the fused protein may be a biotinylated Dengue virus Capsid protein or a West Nile virus Envelope protein DIII—are shown in FIG. 9.

### EXAMPLE 1

### Material and Methods

Construction of pET28aDENVBioCap Plasmid

DENV C gene was amplified from cDNA synthesized from DENV-2 (NGC strain; accession number: M29095; nucleotide 100-438) RNA using SuperScript™ III First-Strand Synthesis System (Life Technologies, USA). Primers Biotin_F (5′-CTAGCTAGCTCCGGCCTGAACGAC-3′) (SEQ ID NO: 1), Biotin_C_F (5′-GACGACGACAAGAGCATGAATAACCAA-3′) (SEQ ID NO: 2), Biotin_C_R (5′-TTGGTTATTCATGCTCTTGTCGTCGTC-3′) (SEQ ID NO: 3), and C_R (5′-CCGCTCGAGTTACGCCATCACTGT-3′) (SEQ ID NO: 4) were used to join biotin acceptor peptide gene (Cull and Schatz, 2000) containing an enterokinase cleavage site with DENV C gene via overlap extension PCR (OE-PCR). Gel-purified PCR products containing the joined fragments were subsequently inserted into expression vector, pET28a (Novagen, Germany) via NheI and XhoI cut sites. 6×His tag and thrombin cleavage site are at the N-terminus of signal peptide followed by enterokinase cleavage site and DENV C protein. DNA sequences of the constructs were confirmed by sequencing.

### Competent Cell Strain Screening

Transformed bacterial colonies of each strain [BL-21 (DE3) and BL-21-CodonPlus] (Agilent Technologies, USA) were picked and grown in 20 ml Luria-Bertani (LB) broth with 30 μg/ml kanamycin antibiotic. When the bacteria absorbance OD₆₀₀ₙₘ reached 0.65, protein expression was induced with 1 mM isopropyl β-D-thiogalactoside (IPTG) overnight at 28° C. After IPTG induction, the bacteria absorbance OD₆₀₀ₙₘ was measured again and 200 μl of equal bacteria density for both strains were used for expression level screening. Bacterial cells were pelleted down with centrifugation at 8,000 rpm for 15 min at 4° C. and resuspended with 100 μl 1× protein sample buffer containing β-mercaptoethanol. The samples were boiled for 10 min with constant vortexing at 1 min interval. Insoluble substances were pelleted down with centrifugation for 2 min at 20,000×g.

### Protein Expression and Extraction

pET28aDENVBioCap plasmid was transformed into BL-21-CodonPlus expression competent cells (Agilent Technologies, USA) and grown on LB agar containing 30 μg/ml kanamycin. Selected clones were cultured in 1 L LB broth (30 μg/ml kanamycin) at 30° C. until absorbance OD₆₀₀ₙₘ of 0.65. Expression of DENV C protein was induced with 1 mM IPTG overnight at 28° C. Bacterial cells were pelleted down with centrifugation at 8,000 rpm for 15 min at 4° C. Pellet was then resuspended in 10 ml resuspension buffer (20 mM Tris, 300 mM NaCl, 0.2% Triton-X, pH 8.0) and pelleted down again at 8,000 rpm for 15 min. The pellet was washed with wash buffer (20 mM Tris, 300 mM NaCl, pH 8.0) before it was resuspended in 30 ml lysis buffer (8 M urea, 20 mM Tris, 300 mM NaCl, 10 mM Imidazole, pH 8.0) containing EDTA-free protease inhibitor (Roche, Switzerland). The mixture was incubated at room temperature for 30 min and the lysate was subsequently clarified by centrifugation at 13,200 rpm for 20 min.

### Nickel-Nitriloacetic Acid (Ni-NTA) Affinity Chromatography

Bacterial lysate containing denatured DENV C protein was incubated with nickel-nitrilotriacetic acid (Ni-NTA) resin (Bio-Rad, USA) for binding in a chromatography column overnight at 4° C. Ten column volume of wash buffer (8 M urea, 20 mM Tris, 300 mM NaCl, 20 mM Imidazole, pH 8.0) was used to wash away non-specific binding proteins. DENV C protein was eventually eluted out with elution buffer (8 M urea, 20 mM Tris, 300 mM NaCl, 500 mM Imidazole, pH 8.0) in ten fractions. Next, all eluates were combined for refolding and dialysis to remove 8 M urea. Briefly, all eluates were pooled into a SnakeSkin dialysis membrane tubing, 3.5k MWCO, (Thermo Scientific, USA) and 0.5% of Tween-20 was added into the samples. The dialysis tubing was incubated in 1 L of 6 M urea for 6-12 hr at 4° C. and 250 ml of 25 mM Tris (pH 8.0) was added into the solution at every 6-12 hr interval. When the final volume reached 3 L, the dialysis tubing was transferred into 2 L of 20 mM Tris (pH 8.0) for 6 hr. Refolded DENV C proteins were collected from the dialysis tubing.

### Ion Exchange Chromatography

Ion exchange chromatography column, Resource™ Q/S 1 ml-packed size column (GE Healthcare, UK), was connected to fast protein liquid chromatography (FPLC) system. The column was equilibrated with ten column volumes of 20 mM Tris (pH 8.0) until the UV baseline and conductivity are stable. Refolded DENV C protein was injected into the column and the flow rate was set to 0.5 ml/min. After the sample passed through the column, ten column volumes of 20 mM Tris (pH 8.0) was used to wash away all the unbound proteins. Ionically-bound proteins were eluted out with increasing concentration of sodium chloride to a final concentration of 1 M (100%). All the fractions were analyzed via ELISA to determine the presence of biotinylated C protein.

### Size Exclusion Chromatography

Superdex 75 10/300 GL chromatographic separation column (GE Healthcare, UK) was connected to FPLC system. The column was washed with five column volumes of MiIIiQ™ water and then equilibrated with two column volumes of 1×phosphate buffered saline (PBS) (pH 7.2). Samples were injected into the column and the flow rate was set to 0.25 ml/min. All the eluates were analyzed via ELISA to determine the presence of biotinylated C protein.

### Enzyme Linked Immunosorbent Assay (ELISA)

To determine whether the expressed protein is biotinylated or not, 50 μl of samples were added into the wells of MaxiSorp plate (eBioscience, USA) in triplicate for coating overnight at 4° C. After washing with 1×0.1% PBST, 150 μl blocking buffer (4% bovine serum albumin) was added into each well and incubated for another hour at room temperature. Next, 150 μl streptavidin-HRP enzyme conjugates (1:5000 dilution) was added and incubated for 1 hr. The plate was washed with 1×PBST three times to remove unbound conjugates and then 100 μl substrate solution, tetramethyl benzidine [(TMB) (Promega, USA)], was added for development. To stop the reaction, 50 μl of 0.5 M H₂SO₄ solution was added. The absorbance was measured at 450 nm.

### Product Analysis

Samples collected from flow through, wash, and eluates were analyzed by SDS-PAGE and Western blot. Twelve % Tris-tricine polyacrylamide denaturing gel was used to separate proteins in the samples and subsequently stained with Coomassie blue for detection. The presence of biotinylated DENV C protein was confirmed by Western blot via two different approaches. First, the identity of DENV C protein was determined with anti-His antibody. Briefly, separated proteins were transferred from polyacrylamide gel onto a PVDF membrane using iBlot® Dry Blotting System (Life Technologies, USA). Blocking was done with 5% skimmed milk for 1 hr at room temperature. Next, the membrane was incubated with 0.1 μg/ml mouse anti-His antibody (Qiagen, Germany) overnight at 4° C. The membrane was then washed with 1×TBST and incubated with 0.1 μg/ml goat anti-mouse secondary antibody conjugated with HRP (Thermo Scientific, USA) for 1 hr at room temperature. After washing with 1×TBST, the membrane was developed using SuperSignal® West Pico/Dura/Femto chemiluminescent substrate (Thermo Scientific, USA).

For the second approach, DENV C protein was detected directly using streptavidin conjugated with HRP. After transferring the samples onto a PVDF membrane, it was blocked with 4% bovine serum albumin (BSA) for 1 hr at room temperature. The membrane was then incubated with HRP-conjugated streptavidin (Millipore, USA) for another hour at room temperature. Subsequently, the membrane was washed thoroughly with 1×PBST for 1 hr at room temperature and developed with chemiluminescent substrate.

### Sample Preparation for Mass Spectrometry

Purified protein was electrophoresed through SDS-PAGE using 12% Tris-tricine polyacrylamide denaturing gel and stained with Coomassie blue. The background of Coomassie-stained gel was removed with destaining solution (40% methanol, 10% glacial acetic acid, 50% distilled H2O). DENV C protein-corresponding band was excised from the gel and kept in eppendorf tube containing distilled water. Samples were submitted to Dr. Lim Yong Pin (Department of Biochemistry, NUS) for matrix-assisted laser desorption/ionization-time of flight (MALDI-TOF) mass spectrometry analysis.

### Biotinylated Protein Binding Assay

The binding affinity of purified biotinylated DENV C protein was tested using streptavidin magnetic beads (GE Healthcare, UK) according to the manufacturer's protocol. Briefly, samples were mixed with the streptavidin magnetic beads and incubated for 1 hr with gentle mixing at 4° C. Unbound proteins were removed with wash buffer while biotinylated proteins were eluted out with elution buffer provided in the kit. Eluted proteins were analyzed by ELISA to confirm the biotinylation.

### DENV C Protein Functional Assay

Pure Sec3 protein [(Abnova, Taiwan) (known interacting partner of flavivirus C protein (Bhuvanakantham, Li, Tan and Ng, 2010))] was added into the wells of MaxiSorp plate (eBioscience, USA) for coating overnight at 4° C. After washing with 1×PBST, blocking buffer (4% bovine serum albumin) was added into each well and incubated for another hour. Purified DENV C protein was added into the well for binding at 37° C. for 1 hr. Next, streptavidin-HRP enzyme conjugates was added and incubated for another hour at 37° C. After washing three times with 1×PBS, 100 μl TMB substrate (Promega, USA) was added for development and 50 μl 0.5 M H₂SO₄ solution was added to stop the reaction when necessary. The absorbance was measured at 450 nm.

### Results

Engineering of Biotin Acceptor Peptide (BAP) into Full-Length DENV Capsid (C) Plasmid, pET28aDENVBioCap

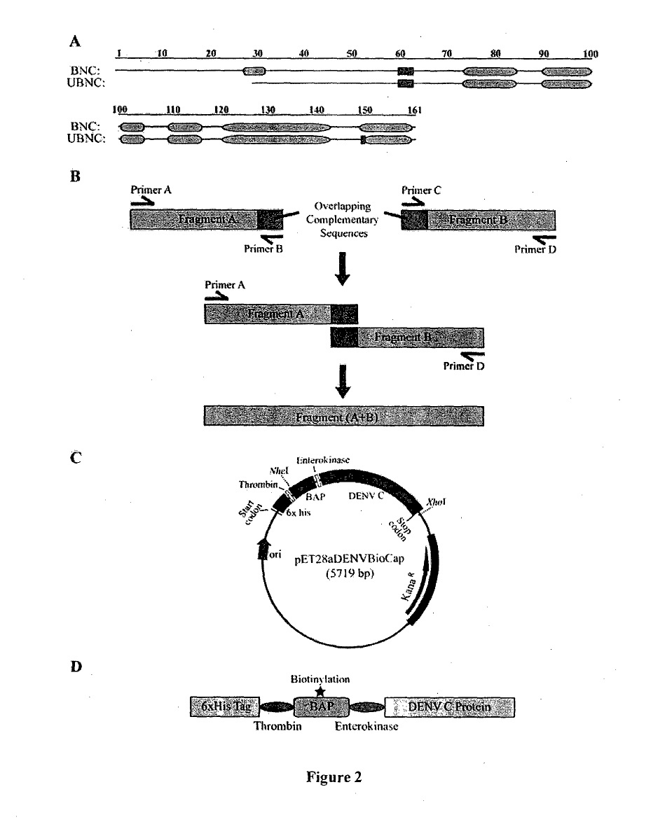

To avoid non-specific biotinylation, we engineered a biotin acceptor peptide (BAP) at the N-terminus of DENV C protein. Biotin is a very small molecule (molecular weight=244.31) so it is unlikely to affect the structure and function of the protein. To ensure that the BAP will not result in any steric hindrance to the protein conformation, a secondary structure prediction was performed. As shown in FIG. 2A, the predicted secondary structures of DENV C protein with and without BAP do not differ from each other.

To clone the full-length DENV C protein, nucleotides 100 to 438 from DENV-2 genome (GeneBank accession number: M29095) was amplified (Fragment B in FIG. 2B), whereas the BAP sequence (Cull and Schatz, 2000) was synthesized together with an enterokinase cleavage site at the C-terminus (Fragment A in FIG. 2B). Both fragments were joined together through overlapping extension-PCR (OE-PCR) method as illustrated in FIG. 2B. The schematic representation of the final construct is shown in FIGS. 2C and 2D.

The final ligated product was then cloned into bacterial expression vector, pET28 which consisted of a 6×His tag and a thrombin cleavage site in the upstream of the multiple cloning sites. Hence, the recombinant full-length DENV C construct (pET28aDENVBioCap) contained 2 tags (6×His tag and BAP) at the N-terminus and 2 different enzyme cleavage sites (thrombin cleavage site and enterokinase cleavage site) for tag removal when necessary.

Five successfully transformed bacterial colonies were picked for colony PCR screening and DNA sequencing was performed to verify the constructs. The final DNA and protein sequence were shown in Suppl. Table 1. To further support our site-specific biotinylation strategy, WNV domain III (DIII) protein which has similar molecular weight as the C protein, was also engineered with a BAP concurrently via the same procedure. DENV C and WNV DIII proteins without BAP were also constructed for comparison purposes.

### Optimal Expression Competent Bacterial Strain for DENV C Protein

To express DENV C protein, an optimal expression competent bacterial strain is requisite. We detected 14 rare codons in the full-length DENV C protein DNA sequence (Suppl. Table 2). This raised a concern for protein expression because 12% of the total 115 codons are rare codons. To screen for the optimal bacterial expression competent cell, pET28aDENVBioCap plasmid was transformed into BL-21 (DE3) and BL-21-CodonPlus. BL-21 (DE3) is a common bacterial strain for high expression of recombinant protein while BL-21-CodonPlus is a bacterial strain specifically engineered for expression of protein with rare codons.

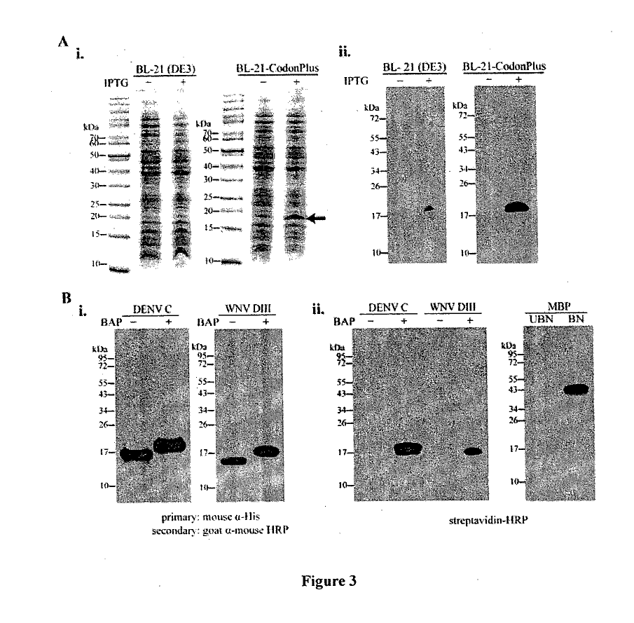

The expression of DENV C protein was indeed much higher in BL-21-CodonPlus strain. As indicated in FIG. 3, an obvious band corresponding to the recombinant full-length DENV C protein was detected in the lysate of IPTG-induced CodonPlus strain but not in the BL-21 (DE3) strain (FIG. 3Ai). Western blotting with anti-His antibody revealed that DENV C protein was expressed in both BL-21 (DE3) and BL-21-CodonPlus strains because C protein-corresponding bands were detected in the lysates of both strains (FIG. 3Aii). Nonetheless, the observed bands were much thicker in BL-21-CodonPlus strain as compared to BL-21 (DE3) strain. This demonstrated that DENV C protein expression level was much higher in BL-21-CodonPlus strain under the same condition. As for WNV DIII protein, the expression was good enough in normal BL-21 (DE3) strain (data not shown).

### Discovery of Endogenous Biotinylation in BL-21 Strains

As comparing DENV C protein with and without BAP via Western-blot using anti-His antibody, bands were observed in all the lanes for DENV C and WNV DIII proteins (FIG. 3Bi). As shown in FIG. 3Bi, recombinant proteins with BAP have higher molecular weight than those without BAP. This also indicated that all the DENV C and WNV DIII proteins with and without BAP were expressing well in the BL-21 strains.

Besides detecting the proteins of interest using anti-His antibody, streptavidin-horseradish peroxidase (HRP) was also employed to detect the presence of biotinylated protein. Surprisingly, we found that our proteins of interest were biotinylated even before any in vitro biotinylation process. As shown in FIG. 3Bii, thick bands can be observed in the BAP-containing DENV C and WNV DIII proteins when streptavidin-HRP antibody was used. Similar bands were not detected in DENV C and WNV DIII proteins without BAP. Commercially-available biotinylated (BN) and unbiotinylated (UBN) maltose-binding protein [(MBP) (GeneCopoeia, USA)] were used as positive and negative controls for biotinylation, respectively. Similarly, band was only seen in the biotinylated MBP Lane. This result suggested that DENV C and WNV DIII proteins with BAP were biotinylated endogenously in BL-21 strains.

We postulated that bacterial BL-21 strains may contain biotin holoenzyme synthetase BirA gene that encodes for biotin ligase protein in their genome. To unveil the mystery as to why biotinylation could occur endogenously, BirA gene sequence was analyzed using BLAST software and the blasting result showed that BL-21 strains indeed possess BirA gene in their genome (Supplementary Table 3). It was also reported that biotin molecules were present in the Luria-Bertani (LB) broth (Tolaymat and Mock, 1989). As a result, proteins engineered with BAP could be directly expressed and biotinylated in BL-21 strains without the extra in vitro enzymatic biotinylation step.

### Optimized Sequential Purification Protocol for Full-Length C Proteins

After confirming the expression and biotinylation of the protein of interest, large scale production of bacterial culture was carried out. Initial attempt was to perform all the extraction and purification steps in native form so that the protein structure could be preserved without the need of refolding. However, the extraction of DENV C protein from the bacterial cells was not effective in native condition. There were still considerably huge amount of DENV C protein trapped in the pellet (data not shown). Therefore, 8 M urea was used to lyze the bacterial cells and affinity his-tag chromatography purification was first performed under denaturing condition.

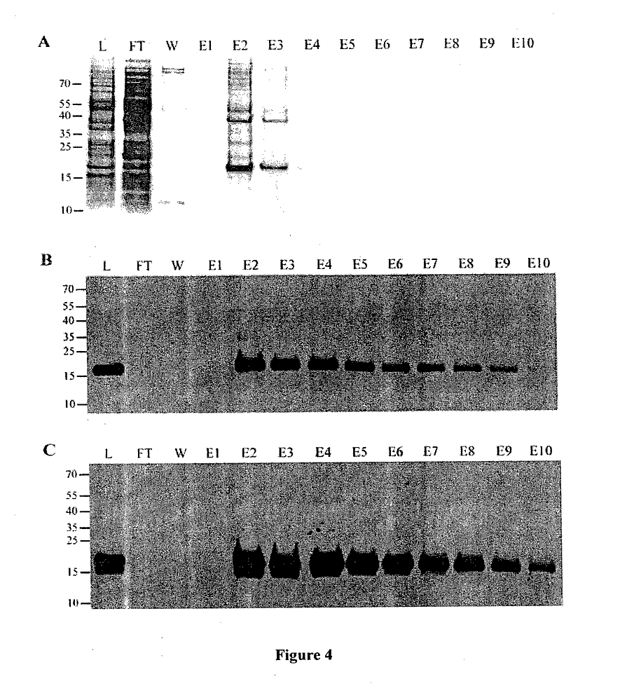

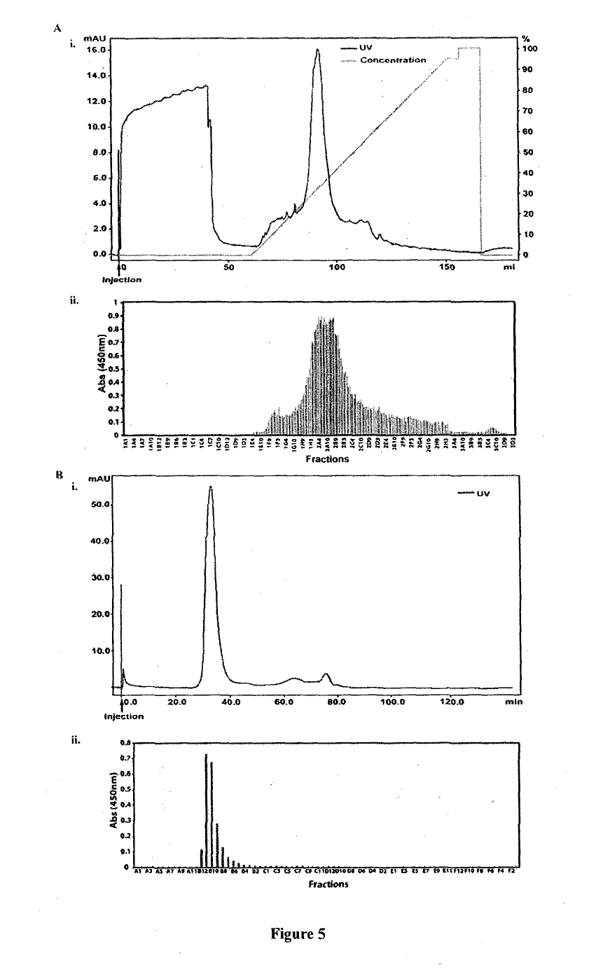

The bacterial cell lysate with biotinylated DENV C protein was incubated with nickel-nitrilotriacetic acid (Ni-NTA) resin to purify His-tagged proteins. Recombinant full-length DENV C protein contained 6×His tag at the N-terminus bound to the resin and unbound proteins were removed during washing with 20 mM imidazole. To ensure most of the unbound proteins were washed away, ten column volumes of wash buffer were used. The bound proteins were eluted out with 500 mM imidazole. However, many non-specific bands were still observed in the eluates of DENV C protein (FIG. 4A), especially in the second eluate fraction. Nonetheless, Western-blot result confirmed that most of the DENV C proteins were eluted out starting from fraction E2 until E10 (FIGS. 4B and 4C).

Partially purified DENV C proteins from eluates E2 to E10 were pooled together for step-wise dialysis and concurrent refolding. Due to the hydrophobic C-terminus of DENV C protein, the protein easily aggregated resulting in major loss during refolding and dialysis. To prevent protein aggregation and to enhance efficient refolding, 0.05% of Tween-20 was added to the samples (Krupakar et al., 2012). With this addition of detergent in the samples, protein aggregation was reduced significantly. No obvious precipitation was observed and the solution was clear after dialysis and refolding. This partially purified DENV C protein was then subjected to second round of purification using ion exchange chromatography-fast protein liquid chromatography system (IEX-FPLC).

Theoretically, DENV C protein is a positively-charged protein so cation-exchanger column, Resource MonoS, should be used. However, we found that biotinylated DENV C protein was not eluted out at a specific concentration of sodium chloride (NaCl). Miniature amount of the protein was eluted out slowly in an increasing gradient manner and it reached plateau at 70 mM of NaCl (Suppl. FIG. 1). This indicated that Resource MonoS column is not suitable for separating DENV C protein from the contaminants.

Counter intuitively, we were able to obtain better separation when we used anion-exchanger, Resource MonoQ, column (FIG. 5Ai). This could be due to the presence of BAP in the DENV C protein conferring its ability to bind to positively charged beads. As shown in FIG. 5A, biotinylated full-length DENV C protein bound perfectly to Resource MonoQ column as no biotinylated proteins were detected by ELISA in the flow through although there was high UV absorbance detected in those fractions. During elution, one high peak was detected when the NaCl concentration reached approximately 350 mM (35%) while there was another small peak detected at the elution concentration of 100 mM of NaCl (10%). These peaks were confirmed to be biotinylated proteins as detected by ELISA using streptavidin-HRP antibody. This result showed that the second purification step managed to further separate most of the contaminants from DENV C protein.

To avoid any other contaminants that may have similar charge as biotinylated DENV C protein, eluates corresponding to the high UV absorbance peak after IEX-FPLC were injected into size-exclusion chromatography (SEC), Superdex 75 10/300 GL column. It was previously reported that SEC could not be used to estimate the molecular weight of DENV C protein because there were many unspecific interaction between the gel matrix and C protein (Jones, Ma, Burgner, Groesch, Post and Kuhn, 2003). Nevertheless, it is still a good method to further isolate the proteins of interest from other possible residual contaminants based on the molecular weight differences. After size-exclusion chromatography, three peaks were observed in the chromatogram (FIG. 5Bi). The first peak was identified to be the biotinylated DENV C protein as the fractions detected with high absorbance in ELISA using streptavidin-HRP antibody coincided with the first peak in the elution profile (FIG. 5Bii). The identity of this highly purified protein was further validated with matrix-assisted laser desorption/ionization-time of flight (MALDI-TOF) mass spectrometry analysis (Suppl. 351 Table 4).

With this optimized sequential purification protocol, we were able to obtain 0.8-1.0 mg of highly purified biotinylated non-truncated, full-length DENV C protein from 1 L of IPTG-induced bacterial culture repeatedly.

### High In Vivo Biotinylation Efficiency in BL-21 Strains

To compare the biotinylation efficiency of our strategy, the same amount of purified biotinylated DENV C, WNV DIII and MBP proteins were coated on the ELISA plate and streptavidin-HRP antibody was used for detection. Assuming that the coating efficiencies are the same among all three proteins, the absorbance detected via direct ELISA should be proportional to the number of coated proteins and inversely proportional to the power of ⅔ of its molecular size. The larger the protein, the lesser the proteins can be coated on the plate.

$$Absorbance ∝ \frac{Protein Concentration}{{( Protein Size )}^{2 / 3}}$$

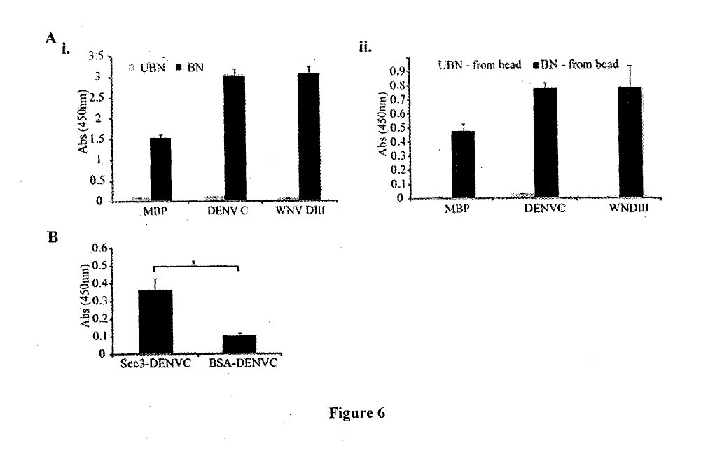

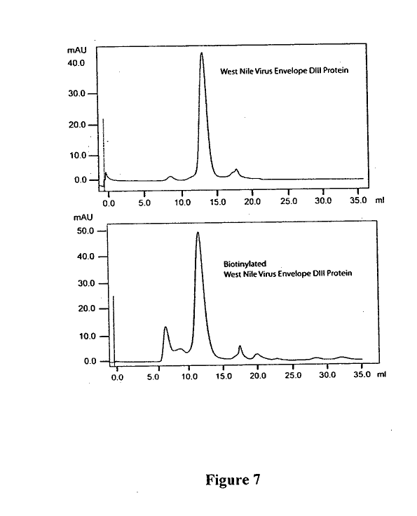

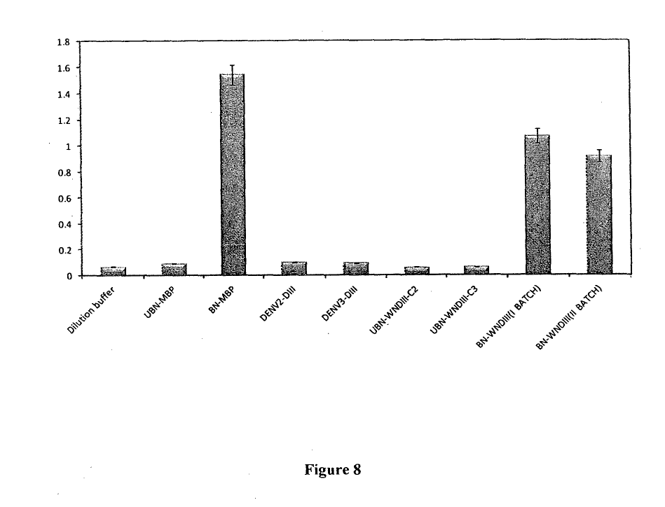

The molecular size of biotinylated MBP is about two times larger than biotinylated DENV C and WNV DIII proteins. Assuming that one protein only carries one biotin which will bind to one streptavidin-HRP only, the absorbance of MBP should be two times lower than our purified proteins for the same protein concentration. As shown in FIG. 6i, the absorbance of purified biotinylated DENV C and WNV DIII proteins indeed showed approximately two times higher the absorbance as compared to commercial biotinylated MBP. The same phenomenon was also observed when the biotinylated proteins were streptavidin-captured by streptavidin-magnetic beads and detected via direct ELISA (FIG. 6Aii). The absorbance of eluted biotinylated DENV C protein and WNV DIII protein were two times higher than that of eluted biotinylated MBP. No significant absorbance was detected for unbiotinylated C and DIII proteins.

This result further supported that engineering an additional BAP on a protein could result in site-specific endogenous biotinylation with high efficiency in Escherichia coli BL-21 strains, without the need of an extra in vitro enzymatic or chemical conjugation step. Our strategy could produce biotinylated proteins that were as good quality as the manufactured biotinylated MBP.

### Purified Non-Truncated, Full-Length DENV C Protein is Functional

It is essential to ensure that the purified proteins are functional before they are used for any further studies. According to our recent findings, WNV and DENV C proteins were found to interact with human Sec3 exocyst protein (Bhuvanakantham, Li, Tan and Ng, 2010). Thus, we examined the functionality of the purified DENV C protein by revisiting the interaction between biotinylated DENV C protein and Sec3 protein using ELISA. Pure Sec3 protein was coated overnight on ELISA plate and purified biotinylated DENV C protein was used as the probe. Bound DENV C proteins were detected with streptavidin-HRP antibody. As shown in FIG. 6B, statistically significant absorbance was detected in Sec3 protein-coated wells but not in BSA-coated wells. Thus, our purified DENV C protein did interact with Sec3 protein. This corroborated that our purified biotinylated full-length DENV C protein is functional and can be used for structural and molecular studies.

### High-Throughput Screening of the Interacting Partners of Dengue Virus Capsid Protein

Dengue virus (DENV) is a positive-stranded RNA virus. Based on the current understanding of flaviviral replication cycle, host cell nucleus is not involved during the transcription, translation and assembly of virus. This traditional notion is challenged when capsid (C) protein is found to localize in the infected cell nucleus. Since then, the role of DENV C protein in the nucleus has been an intriguing mystery for researchers to unveil. Understanding the roles of C protein in the nucleus will not only completes the missing puzzle of flaviviral replication cycle, but also provides insights for novel anti-viral strategies development.

Using the purified biotinylated full-length DENV C protein of the present invention, high-throughput screening (HTS) of novel interacting partners of DENV C protein is made possible. ProtoArray® Human Protein Microarray PPI Complete Kit (Life Technologies, USA) was employed for HTS study of novel DENV C protein-interacting partners. This protein microarray contains 9400 purified human proteins printed in duplicate on the nitrocellulose-coated glass slide. The purified human proteins encompass various cellular kinases, enzymes, nuclear proteins and signalling proteins tagged with glutathione S transferase (GST) or 6×His. Purified biotinylated DENV C protein was used as probe and streptavidin-Alexa Fluor® 647 antibody is chosen as the detection reagent as it yields very good signal to noise ratio.

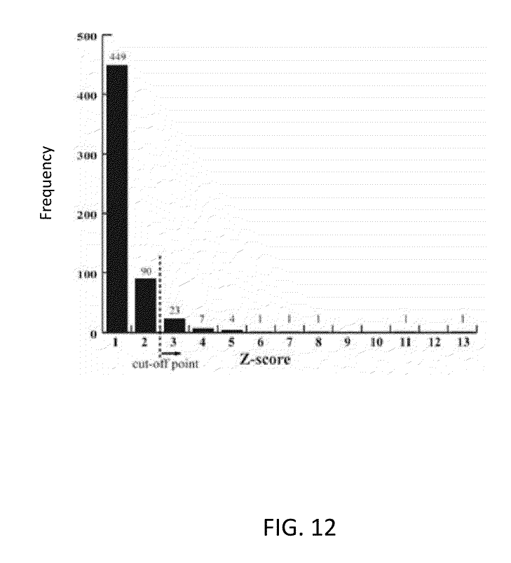

There were 578 potential DENV C protein-binding partners detected to have z-score value above 1 (FIG. 12).

Z-score value of 3 was chosen to be the cut-off point for statistically significant potential interactors. Z-score indicates how far and in what direction the sample's value deviates from the distribution's mean. After filtering some of the proteins without known functions and with biotin-binding property, 31 significant potential interacting partners were identified for DENV C protein as shown in the table below.

Significant Potential Interacting Partners of DENV C Protein

Scanned images of the microarray are analyzed using GenePix® Pro software to determine the pixel intensity and the data acquired are further analyzed via Protoarray® Prospector software to identify potential interacting partners of DENV C protein. Z-score cut-off value of 3 is chosen to be the statistically significant interactors.

| | | | Signal | | | |
|---|---|---|---|---|---|---|
| Identity | Symbol | Database ID | Intensity | CV | Z-score | Function |
| Cortactin | CTTN | NM_138565.1 | 3076.5 | 0.03517 | 13.41837 | Regulate and organize actin |
| | | | | | | cytoskeleton |
| SLAIN motif family, | SLAIN2 | BC031691.2 | 2569.5 | 0.04981 | 11.08491 | Control microtubule growth and |
| member 2 | | | | | | organization |
| Immunoglobulin | IGBP1 | NM_001551.1 | 1938 | 0.02043 | 8.17845 | Binds to surface IgM receptor |
| (CD79A) binding | | | | | | and may involve in the |
| protein 1 | | | | | | activation signal transduction |
| | | | | | | pathway |
| Choline kinase | CHKA | NM_001277.1 | 1738 | 0.03743 | 7.25795 | Catalyzes the phosphorylation of |
| alpha | | | | | | ethanolamine and plays a role |
| | | | | | | in phosphatidylcholine |
| | | | | | | biosynthesis. |
| DIM1 | DIMT1 | NM_014473.2 | 1666.5 | 0.26943 | 6.92887 | Dimethylates 18S rRNA in the |
| dimethyladenosine | | | | | | 40S particle |
| transferase 1 | | | | | | |
| homolog | | | | | | |
| (S. cerevisiae) | | | | | | |
| Ubiquitin- | UBE2S | BC004236.2 | 1353.5 | 0.00366 | 5.4883 | Catalyzes ‘Lys-11’-linked |
| conjugating | | | | | | polyubiquitination on the |
| enzyme E2S | | | | | | anaphase promoting complex/ |
| | | | | | | cyclosome (APC/C) |
| | | | | | | substrates |
| AF4/FMR2 family, | AFF4 | BC025700.1 | 1251 | 0.06896 | 5.01654 | Regulates transcriptional activity |
| member 4 | | | | | | |
| WAS/WASL | WIPF1 | BC002914.1 | 1159 | 0.02562 | 4.59312 | Control the organization of actin |
| interacting protein | | | | | | cytoskeleton |
| family, member 1 | | | | | | |
| Aurora kinase A | AURKA | BC006423.1 | 1158 | 0.04519 | 4.58851 | Cell cycle-regulated kinase |
| | | | | | | regulates microtubules |
| | | | | | | formation and stabilizes |
| | | | | | | spindle poles during mitosis |
| RAD51 associated | RAD51AP1 | BC016330.1 | 1143 | 0.00990 | 4.51948 | Involved in common DNA |
| protein 1 | | | | | | damage response pathway |
| Additional sex | ASXL1 | BC064984.1 | 1072 | 0.04485 | 4.1927 | Regulates transcriptional activity |
| combs like 1 | | | | | | |
| (Drosophila) | | | | | | |
| CDKN2A interacting | CDKN2AIP/ | BC022270.1 | 1031.5 | 0.13642 | 4.0063 | Activates p53/TP53 via |
| protein | CARF | | | | | CDKN2A-dependent and |
| | | | | | | CDKN2A-independent |
| | | | | | | pathways |
| Calcium channel, | CACNB1 | NM_000723.3 | 1017.5 | 0.12162 | 3.94187 | Increases peak calcium current, |
| voltage-dependent, | | | | | | modulates G protein inhibition, |
| beta 1 subunit | | | | | | and shifts the voltage- |
| | | | | | | dependent activation and |
| | | | | | | inactivation. |
| Spermidine/ | SAT2 | NM_133491.2 | 1008.5 | 0.00351 | 3.90044 | Catalyzes the acetylation of |
| spermine N1- | | | | | | polyamines |
| acetyltransferase | | | | | | |
| family member 2 | | | | | | |
| Eukaryotic | EIF1AX | NM_001412.2 | 997 | 0.15461 | 3.84751 | Enchances ribosome |
| translation initiation | | | | | | dissociation into subunits and |
| factor 1A, X-linked | | | | | | stabilizes the binding of Met- |
| | | | | | | tRNA(I) to 40S ribosomal |
| | | | | | | subunits |
| Signal recognition | SRP19 | BC010947.1/ | 971.5/ | 0.34573/ | 3.73015/ | Mediates the assembly of signal |
| particle 19 kDa | | NM_003135.1 | 818 | 0.16770 | 3.02367 | recognition particle |
| Calcium/ | CAMK2A | NM_171825.2 | 969 | 0.11238 | 3.71865 | Involved in hippocampal long- |
| calmodulin- | | | | | | term potentiation by switching |
| dependent protein | | | | | | calmodulin-dependent activity |
| kinase II alpha | | | | | | to calmodulin independent |
| Piccolo (presynaptic | PCLO | BC001304.1 | 929 | 0.02740 | 3.53455 | Involved in the organization of |
| cytomatrix protein) | | | | | | synaptic active zone and |
| | | | | | | synaptic vesicle trafficking |
| Nuclear speckle | NSRP1 | NM_032141.1 | 927.5 | 0.16544 | 3.52764 | Mediates pre-mRNA alternative |
| splicing regulatory | | | | | | splicing regulation |
| protein 1 | | | | | | |
| NIMA (never in | NEK7 | NM_133494.1 | 918.5 | 0.19169 | 3.48622 | Controls initiation of mitosis |
| mitosis gene a)- | | | | | | |
| related kinase 7 | | | | | | |
| Checkpoint | CHES1/ | NM_005197.2 | 918 | 0.27730 | 3.48392 | Transcriptional repressor |
| suppressor 1/ | FOXN3 | | | | | involved in DNA damage- |
| Forkhead box N3 | | | | | | inducible cell cycle arrests at |
| | | | | | | G1 and G2 phases |
| Rtf1, Paf1/RNA | RTF1 | NM_015138.2 | 882.5 | 0.02484 | 3.32053 | Regulates transcriptional activity |
| polymerase II | | | | | | |
| complex | | | | | | |
| component, | | | | | | |
| homolog | | | | | | |
| (S. cerevisiae) | | | | | | |
| Microtubule- | MAP2 | NM_031845.1 | 875 | 0.03879 | 3.28601 | Function unclear. Thought to be |
| associated protein | | | | | | involved in microtubule |
| 2 | | | | | | assembly and stabilizing the |
| | | | | | | microtubules against |
| | | | | | | depolymerization |
| Potassium channel | KCTD18 | NM_152387.2 | 856 | 0.02313 | 3.19857 | Molecular determinants for |
| tetramerisation | | | | | | subfamily-specific assembly of |
| domain containing | | | | | | alpha-subunits into functional |
| 18 | | | | | | tetrameric voltage-gated |
| | | | | | | potassium channels |
| Cyclin B3 | CCNB3 | NM_033671.1 | 853.5 | 0.05385 | 3.18706 | Involved in cell cycle control |
| Eukaryotic | EIF1AY | BC005248.1 | 852.5 | 0.12691 | 3.18246 | Enchances ribosome |
| translation initiation | | | | | | dissociation into subunits and |
| factor 1A, Y-linked | | | | | | stabilizes the binding of Met- |
| | | | | | | tRNA(I) to 40S ribosomal |
| | | | | | | subunits |
| Vaccinia related | VRK1 | NM_003384.1 | 845 | 0.17406 | 3.14794 | Serine/Threonine kinase that is |
| kinase 1 | | | | | | involved in regulating cell |
| | | | | | | proliferation and prevent the |
| | | | | | | interaction between p53/ |
| | | | | | | TP53 and MDM2 |
| Protein kinase C, | PRKCI | NM_002740.1 | 841 | 0.00504 | 3.12953 | Calcium-independent and |
| iota | | | | | | phospholipid-dependent |
| | | | | | | serine/threonine kinase |
| | | | | | | involved various cellular |
| | | | | | | processes |
| WD repeat domain 5 | WDR5 | NM_017588.1 | 840.5 | 0.01598 | 3.12723 | Involved in histone modification |
| | | | | | | and acetylation of histone H3 |
| p53-regulated DDA3 | PSRC1 | NM_032636.2 | 822 | 0.02925 | 3.04208 | Regulates mitotic spindle and |
| (DDA3)/ | | | | | | involved in p53/TP53- |
| proline/serine-rich | | | | | | regulated growth suppression |
| coiled-coil 1 | | | | | | |
| Elongation factor 1 | ELOF1 | NM_032377.2 | 816.5 | 0.00260 | 3.01677 | Maintains proper chromatin |
| homolog (ELF1, | | | | | | structure in actively |
| S. cerevisiae) | | | | | | transcribed regions |

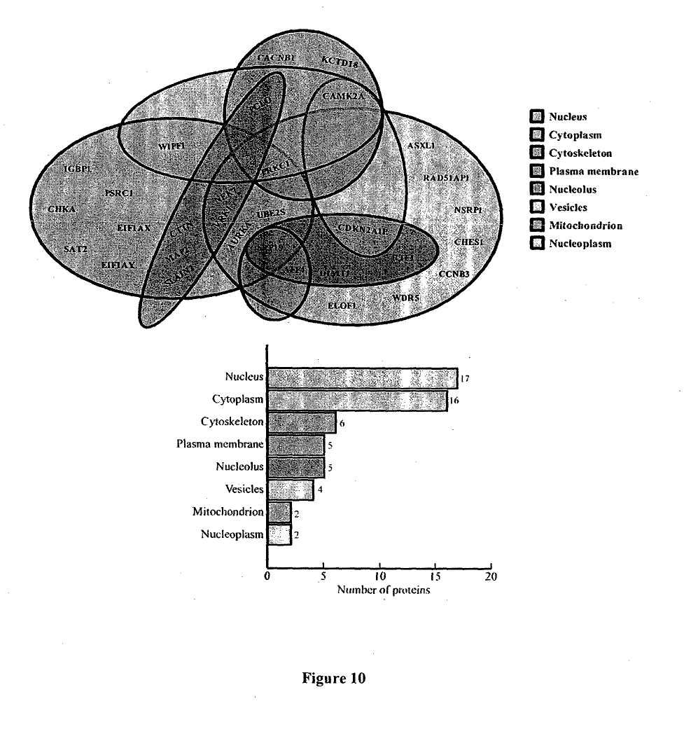

The list of potential interacting partners for DENV C protein was quite well dispersed throughout the cell nucleus and cytoplasm. The total proteins in each cellular compartment are calculated and charted. FIG. 10 illustrates the cellular localization of each individual protein and some of them may localize in more than one cellular compartment. Majority of the DENV C protein-interacting partners localize in the nucleus followed by cytoplasm. Six proteins are related to cytoskeleton while five are found in the nucleolus and plasma membrane. As shown in FIG. 10, approximately 55% of the identified proteins localized in the nucleus while the other half was distributed in the cytoplasm. This was exciting because a comprehensive list of DENV C protein-nuclear interactors involved in the virus replication was identified from this ProtoArray® HTS study.

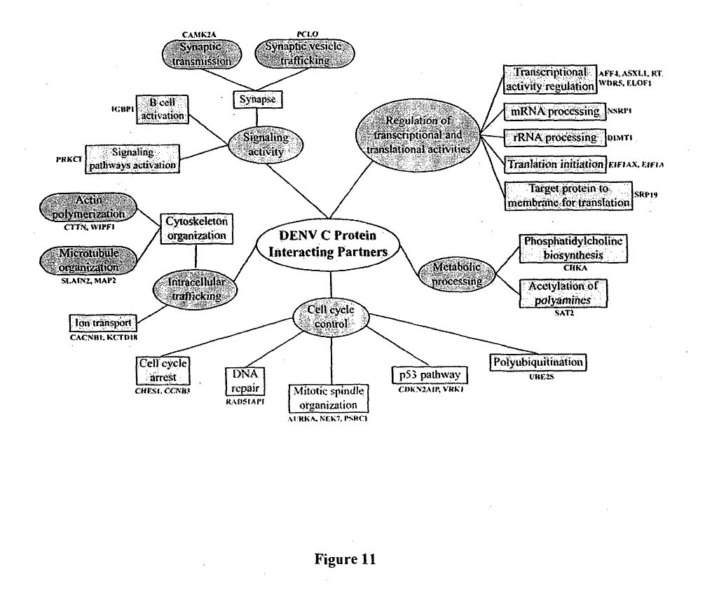

From the list of potential interacting partners, it appeared that DENV C protein may be involved primarily in cell cycle control and regulation of transcriptional and translational activities (FIG. 11). Five proteins (AFF4, ASXL1, RTF1, WDR5, and ELOF1) were involved in the regulation of transcription and four proteins (DIMT1, EIF1AX, EIF1AY, and SRP19) participated in ribosomal biogenesis and protein synthesis. This is a thrilling result because it unravelled potential roles of DENV C protein in the nucleus which is a million-dollar question in flavivirus research. Investigating these novel non-structural roles of flavivirus C protein will provide better understanding of the underlying molecular mechanism of flavivirus infection. This knowledge is essential for more refinement of anti-viral drug designs and development.

### Binding Assay of the Interacting Partners of Dengue Virus Capsid Protein Via ELISA Platform

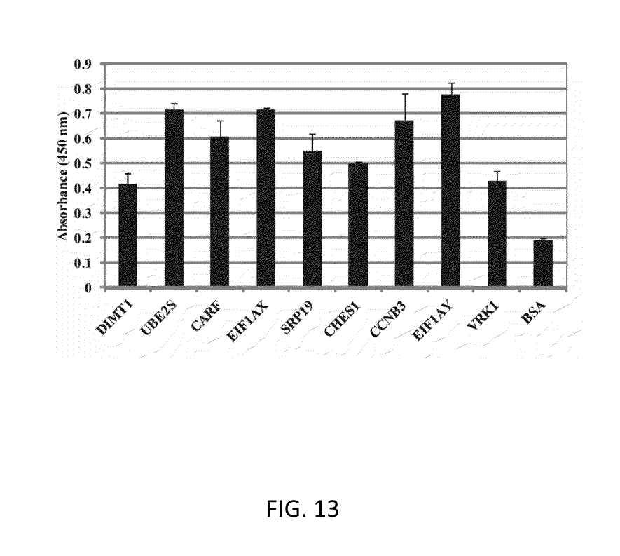

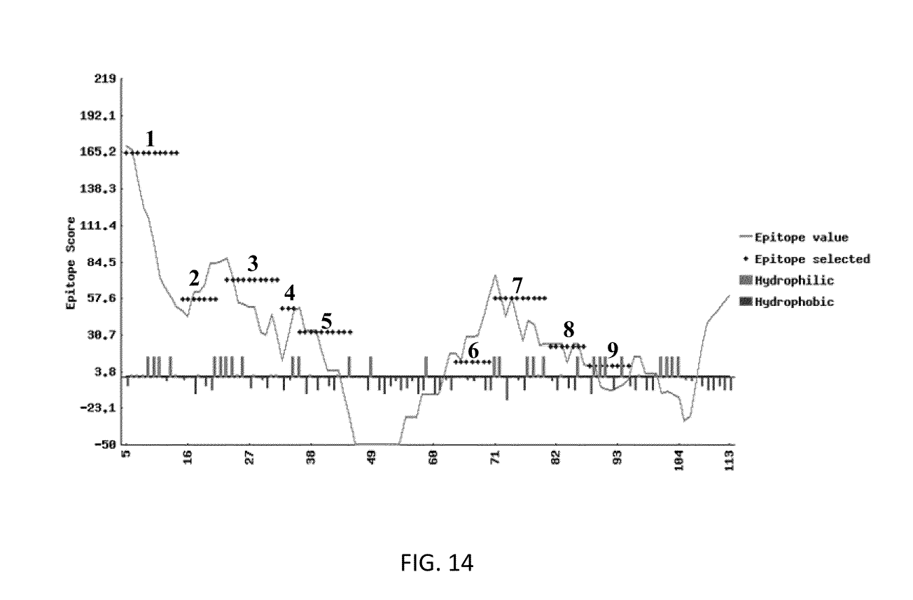

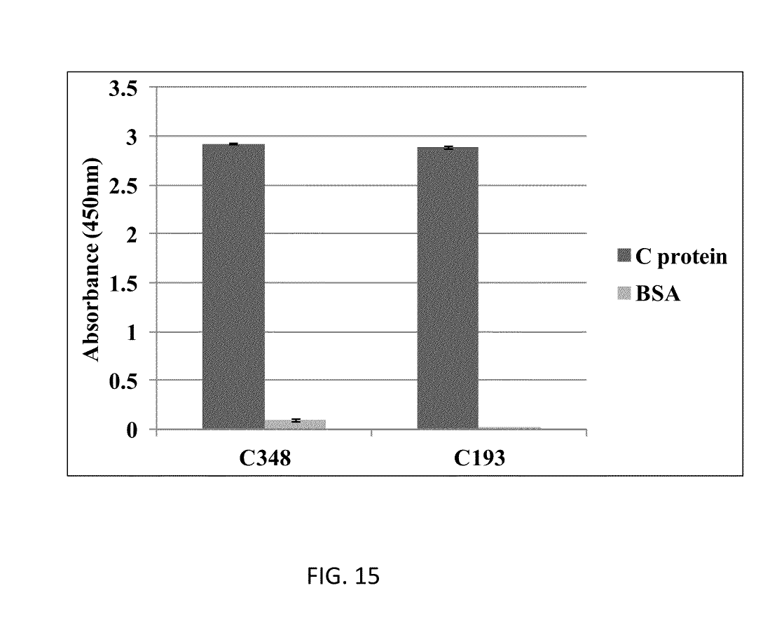

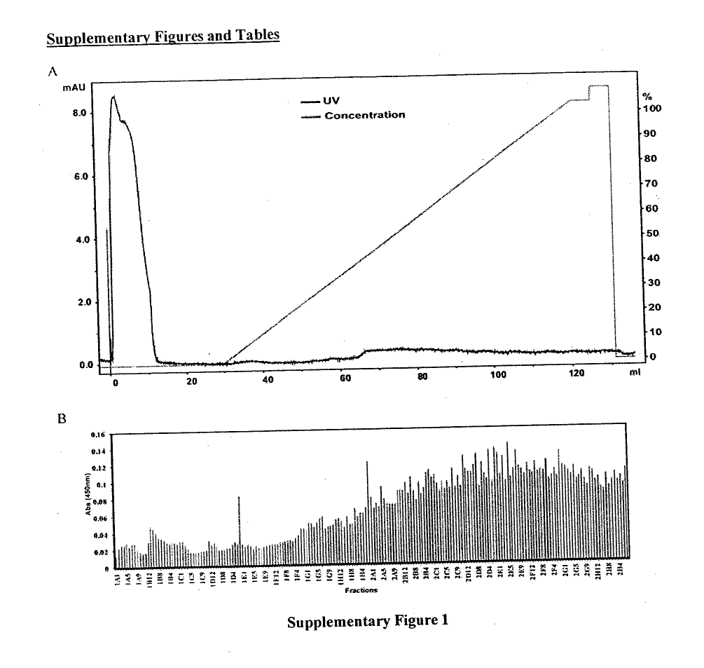

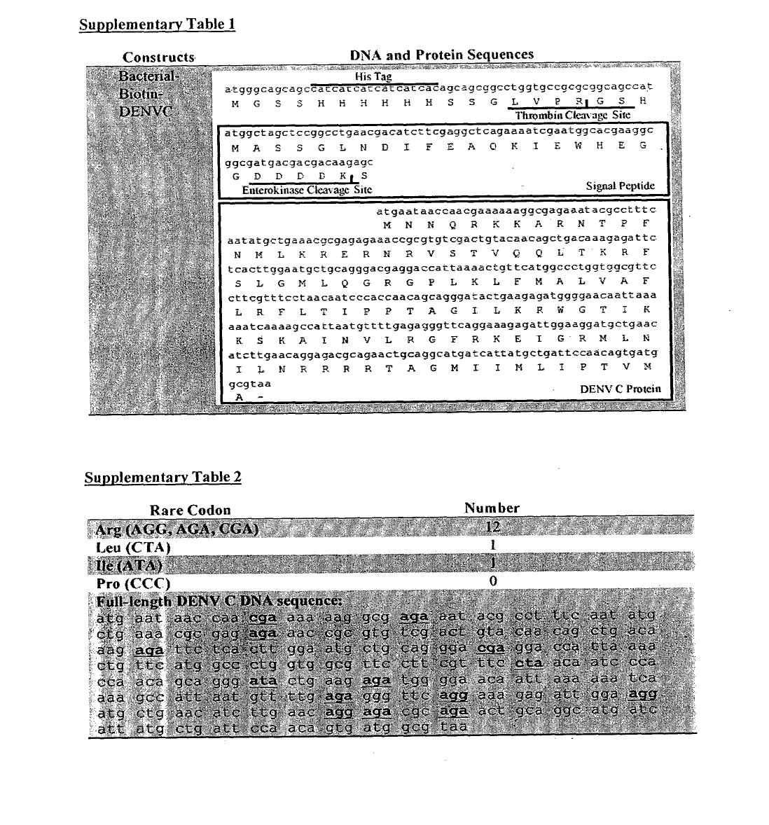

The purified biotinylated full-length Dengue virus capsid protein of the present invention can also be used to verify the binding between DENV C protein and its interacting partners in ELISA platform. To validate the identified potential interacting partners from the protein microarray study, commercially-available pure proteins were purchased and coated on ELISA plate. Biotinylated full-length DENV C protein was used as probe for binding and streptavidin-HRP antibody was used to detect the bound proteins. Four proteins (DIMT1, EIF1AX, EIF1AY, and SRP19) that are related to transcriptional and translational machinery and five proteins pertaining to cell cycle control (UBE2S, CARF, CHES1, CCNB3, and VRK1) were selected for further validation of DENV C protein binding. As shown in FIG. 13, all nine proteins showed significant higher absorbance as compared to bovine albumin serum (BSA) control, implicating that these nine potential interacting partners bound to full-length DENV C protein in an ELISA-based platform.

FIG. 13 shows the verification of the binding between potential interaction partners and Dengue virus capsid protein via ELISA. Commercially-available proteins of the identified interactors of DENV C protein are coated on MaxiSorp 96-well plate in triplicates. Purified biotinylated DENV C proteins are added into the wells for binding and bound proteins are detected via streptavidin-horseradish peroxidase (HRP) secondary antibody. The absorbance is measured at 450 nm. Bovine serum albumin (BSA) is used as negative control for binding.

Therefore, the biotinylated full-length DENV C protein of the present invention is useful to flavivirus laboratories that are studying protein-protein, protein-RNA, and protein-DNA interactions.

### Development of Antibody Against Dengue Virus Capsid Protein

One of the challenges in Dengue virus research is that antibodies against the viral proteins are not available commercially. Most of the laboratories need to raise the antibodies on their own before carrying out any further downstream experiments (Puttikhunt et al., 2009, Wang et al., 2002). Anti-DENV C antibody is one of the missing antibodies in the market. Hence both full-length DENV C protein and anti-DENV C antibody are of commercial values.

Before the present inventors raised anti-C antibody using pure DENV C protein, an antibody generation package from Abmart Inc. was purchased to produce monoclonal antibodies against DENV C peptides. Abmart Inc. was only able to produce monoclonal antibodies against two peptides (MNNQRKKARN (SEQ ID NO: 5) and ERNRVSTVQQ (SEQ ID NO: 7)). Below is a list of predicted epitopes for antibody production:

Epitopes Sequences

| | | | | | |
|---|---|---|---|---|---|
| 1 | MNNQRKKARN | 4 | ARNTPFNMLK | 7 | PPTAGILKRW |
| | (SEQ ID NO: 5) | | (SEQ ID NO: 8) | | (SEQ ID NO: 11) |
| 2 | KRWGTIKKSK | 5 | KSKAINVLRG | 8 | RVSTVQQLTK |
| | (SEQ ID NO: 6) | | (SEQ ID NO: 9) | | (SEQ ID NO: 12) |
| 3 | ERNRVSTVQQ | 6 | RFSLGMLQGR | 9 | RGFRKEIGRM |
| | (SEQ ID NO: 7) | | (SEQ ID NO: 10) | | (SEQ ID NO: 13) |

The epitopes are predicted by Abmart Inc. using their propriety algorithm. An “epitope score” is assigned to each residue based on criteria such as structural features, sequence conservation, hydrophobicity, and solvent exposure. Nine 10-residue fragments with highest overall scores are chosen as epitopes to generate monoclonal antibodies.

The first peptide (MNNQRKKARN) (SEQ ID NO: 5) is the first ten residues from the N-terminus of C protein whereas the second peptide (ERNRVSTVQQ) (SEQ ID NO: 7) is from the 19^{th} residue to the 28^{th} residue. Both epitopes are from the N-terminal region before the first helix (al) structure. Monoclonal antibodies against these two peptides were tested to be able to recognize purified full-length recombinant DENV C protein as shown in FIG. 10. Further characterization of the antibodies is still on-going.

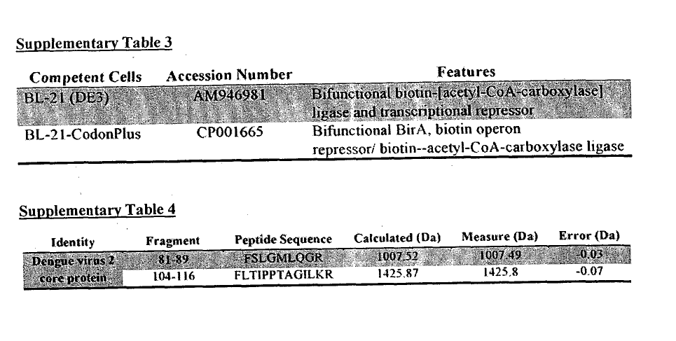

FIG. 15 shows that purified full-length recombinant DENV C protein is recognized by monoclonal antibodies against DENV C peptides. C348 and C193 are monoclonal antibodies against MNNQRKKARN (SEQ ID NO: 5) and ERNRVSTVQQ (SEQ ID NO: 7) peptides, respectively. Purified DENV C protein (100 ng) is coated on the MaxiSorp well in triplicates. Antibodies C348 and C193 are diluted 10,000× and bound antibodies are detected by horseradish peroxidase (HRP)-conjugated anti-mouse secondary antibody. Both monoclonal antibodies can recognize recombinant DENV C protein but not bovine serum albumin (BSA).

### Discussion

To produce biotinylated proteins, we opted for site-specific biotinylation by engineering a biotin acceptor signal peptide (BAP) in the upstream of our proteins. To attach a biotin molecule onto BAP either in vivo or in vitro, bacterial biotin ligase BirA enzyme is required (Chapman-Smith and Cronan, 1999, Cull and Schatz, 2000, Tan et al., 2004, Yang et al., 2004). Instead of performing this biotinylation step in vitro, we found that biotinylation of the BAP-containing proteins occurred endogenously in Escherichia coli BL-21 strains with high efficiency.

The purification of non-truncated, full-length DENV C protein was indeed challenging. Unlike DIII protein (Tan et al., 2010), one single purification step was simply not adequate to produce pure DENV C protein. The presence of rare codons in the C protein sequence necessitates unique expression competent strain for its optimal protein expression. Due to the hydrophobic C-terminus, aggregation easily occurred and most of the expressed proteins were trapped in the inclusion bodies. As such, we had to use 8 M urea to denature the proteins and perform affinity chromatography under denaturing condition. We also managed to solve the aggregation problem during dialysis and refolding by using non-ionic detergent. To obtain highly purified DENV C protein, two more purification steps were included in our protocol, namely IEX and SEC. This sequential purification strategy can produce approximately 1 mg of purified DENV C protein from 1 L of bacterial culture.

This purified non-truncated, full-length DENV C protein is useful for various molecular and structural studies. For instance, it can be used to study the interaction of NS2B-NS3 serine proteinase and C protein. In order to produce mature C protein for encapsidation and assembly, full-length C protein has to be cleaved by NS2B-NS3 protein complex. Thus, it is crucial to identify the binding sides of NS2B-NS3 complex to full-length C protein, which is still unknown. It will in turn lead to the development of novel anti-viral drugs targeting the assembly of virus particles. Besides, it is also interesting to examine whether the cleaved C-terminus plays any unidentified role in the pathogenesis. The crystal structure of DENV NS2B-NS3 protein complex has been resolved (Erbel et al., 2006). However, the crystal structure of full-length DENV C protein is still not available yet, although the NMR structure of partial DENV C protein was obtained (Ma, Jones, Groesch, Kuhn and Post, 2004). We are now taking up the challenge to resolve the 3D structure of full-length DENV C protein using our purified protein.

Ma and co-workers (2004) proposed a model for the interaction between C protein and its viral genome using the partial DENV C protein. However, this model is still not validated and the exact mechanism of encapsidation is still unclear until to date. Investigation of how DENV C protein interacts with its RNA and oligomerizes into nucleocapsid during maturation is vital for better understanding of the pathogenesis. Recent publications also demonstrated that N-terminus of DENV C protein was antigenic in mice study and the first 18 amino acid residues were involved in the virus assembly during maturation (Puttikhunt, Ong-Ajchaowlerd, Prommool, Sangiambut, Netsawang, Limjindaporn, Malasit and Kasinrerk, 2009, Samsa, Mondotte, Caramelo and Gamarnik, 2012). As a result, our purified non-truncated DENV C protein is absolutely desirable for delineating the whole process of encapsidation and virus assembly.

Another difficulty of studying DENV C protein is that anti-C antibody is yet to be available commercially. Most of the laboratories need to raise this antibody on their own before any further downstream experiments (Puttikhunt, Ong-Ajchaowlerd, Prommool, Sangiambut, Netsawang, Limjindaporn, Malasit and Kasinrerk, 2009, Wang et al., 2002). Hence, pure full-length DENV C protein can be obtained simply by enterokinase cleavage and subsequently used to raise antibody in mice or rabbit for laboratory research usage and diagnostic tool. Both full-length DENV C protein and anti-DENV C antibody will be of commercial values.

In conclusion, we have developed an optimized protocol to engineer, express and purify the first biotinylated, full-length DENV C protein. This purified protein is useful for various molecular studies to further understand the underlying mechanism of C protein in pathogenesis. Only by stitching all these missing pieces of the puzzle together, novel antiviral strategies can be rationally designed.

Whilst there has been described in the foregoing description preferred embodiments of the present invention, it will be understood by those skilled in the technology concerned that many variations or modifications in details of design or construction may be made without departing from the present invention.

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art.

For the purposes of this specification, it will be clearly understood that the word comprising means including but not limited to, and that the word comprises has a corresponding meaning.

### REFERENCES

1. Amberg S. M. and Rice C. M. (1999) J Virol, 73, 8083-8094.
2. Bayer E. A. and Wilchek M. (1990) Methods Enzymol, 184, 174-187.
3. Bayer E. A. and Wilchek M. (1990) Methods Enzymol, 184, 138-160.
4. Bhuvanakantham R., Chong M. K. and Ng M. L. (2009) Biochem Bioph Res Co, 389, 63-69. Bhuvanakantham R., Li J., Tan T. T. T. and Ng M. L. (2010) Cellular Microbiology, 12, 453-472.
5. Chapman-Smith A. and Cronan J. E., Jr. (1999) Trends Biochem Sci, 24, 359-363.
6. Chapman-Smith A. and Cronan J. E., Jr. (1999) Biomol Eng, 16, 119-125.
7. Cull M. G. and Schatz P. J. (2000) Methods Enzymol, 326, 430-440.
8. de Boer E., Rodriguez P., Bonte E., Krijgsveld J., Katsantoni E., Heck A., Grosveld F. And Strouboulis J. (2003) Proc Natl Acad Sci USA, 100, 7480-7485.
9. Erbel P., Schiering N., D'Arcy A., Renatus M., Kroemer M., Lim S. P., Yin Z., Keller T. H., Vasudevan S. G. and Hommel U. (2006) Nat Struct Mol Biol, 13, 372-373.
10. Gubler D. J. (2002) Arch Med Res, 33, 330-342.
11. Hase T., Summers P. L., Eckels K. H. and Baze W. B. (1987) Archives of Virology, 92, 273-291.
12. He Y. F., Bao H. M., Xiao X. F., Zuo S., Du R. Y., Tang S. W., Yang P. Y. and Chen X. (2009) Proteomics, 9, 5414-5424.
13. Howarth M. and Ting A. Y. (2008) Nat Protoc, 3, 534-545.
14. Jones C. T., Ma L. X., Burgner J. W., Groesch T. D., Post C. B. and Kuhn R. J. (2003) J Virol, 77, 7143-7149.
15. Krupakar P., Ngo A. M. L. and Ng M. L. (2012) In Miguel Benitez and Aguirre V. (eds) Protein Purification. Nova Science, New York, pp. 147-170.
16. Ma L., Jones C. T., Groesch T. D., Kuhn R. J. and Post C. B. (2004) Proc Natl Acad Sci USA, 101, 3414-3419.
17. Markham K., Bai Y. and Schmitt-Ulms G. (2007) Anal Bioanal Chem, 389, 461-473.
18. Markoff L. (1989) J Virol, 63, 3345-3352.
19. Moreland N. J., Tay M. Y., Lim E., Paradkar P. N., Doan D. N., Yau Y. H., Geifman Shochat S. and Vasudevan S. G. (2010) PLoS Negl Trop Dis, 4, e881.
20. Postel A., Letzel T., Muller F., Ehricht R., Pourquier P., Dauber M., Grund C., Beer M. and Harder T. C. (2011) Anal Biochem, 411, 22-31.
21. Puttikhunt C., Ong-Ajchaowlerd P., Prommool T., Sangiambut S., Netsawang J.,
22. Limjindaporn T., Malasit P. and Kasinrerk W. (2009) Arch Virol, 154, 1211-1221
23. Qi Y. and Katagiri F. (2011) Methods Mol Biol, 712, 21-30.
24. Samsa M. M., Mondotte J. A., Caramelo J. J. and Gamarnik A. V. (2012) J Virol, 86, 1046-1058.
25. Sueda S., Yoneda S. and Hayashi H. (2011) Chembiochem, 12, 1367-1375.
26. Tadano M., Makino Y., Fukunaga T., Okuno Y. and Fukai K. (1989) J Gen Virol, 70 (Pt 6), 1409-1415.
27. Tan L. C., Chua A. J., Goh L. S., Pua S. M., Cheong Y. K. and Ng M. L. (2010) Protein Expr Purif, 74, 129-137.
28. Tan L. P., Lue R. Y., Chen G. Y. and Yao S. Q. (2004) Bioorg Med Chem Lett, 14, 6067-6070.
29. Tolaymat N. and Mock D. M. (1989) J Nutr, 119, 1357-1360.
30. Wang S. H., Syu W. J., Huang K. J., Lei H. Y., Yao C. W., King C. C. and Hu S. T. (2002) J Gen Virol, 83, 3093-3102.
31. Yang J., Jaramillo A., Shi R., Kwok W. W. and Mohanakumar T. (2004) Hum Immunol, 65, 692-699.
32. Yang M. R., Lee S. R., Oh W., Lee E. W., Yeh J. Y., Nah J. J., Joo Y. S., Shin J., Lee H. W., Pyo S. et al. (2008) Cell Microbiol, 10, 165-176.
33. Zhang W., Chipman P. R., Corver J., Johnson P. R., Zhang Y., Mukhopadhyay S., Baker T. S., Strauss J. H., Rossmann M. G. and Kuhn R. J. (2003) Nat Struct Biol, 10, 907-912.

1. A fused protein comprising a moiety and a protein selected from a group consisting of flavivirus structural and non-structural (NS) proteins.

2. The fused protein according to claim 1, wherein the flavivirus structural protein is a flavivirus capsid protein or a flavivirus envelope protein.

3. The fused protein according to claim 1, wherein the moiety is a readily detectable moiety.

4. The fused protein according to claim 3, wherein the moiety is a biotin acceptor signal peptide.

5. The fused protein according to claim 1, wherein the flavivirus is a Dengue virus or a West Nile virus.

6. The fused protein according to claim 5, wherein the biotin acceptor peptide is fused with a West Nile envelope Domain III protein.

7. The fused protein according to claim 5, wherein the biotin acceptor peptide is fused at the N-terminus of the Dengue virus capsid protein.

8. The fused protein according to claim 1, wherein the fused protein is full-length and non-truncated.

9. The fused protein according to claim 1 for use in medicine.

10. An isolated nucleic acid encoding the fused protein according to claim 1.

11. An expression vector comprising the nucleic acid according to claim 10.

12. The expression vector according to claim 11, wherein the expression vector is a plasmid.

13. A host cell comprising the expression vector according to claim 11.

14. An immunogenic composition or vaccine comprising a fused protein according to claim 1.

15. A pharmaceutical composition for treating a flavivirus infection in a patient, the composition comprising a therapeutic effective amount of a fused protein or fragment thereof according to claim 1 and at least one pharmaceutically acceptable carrier, excipient or diluent.

16. A method for treating a flavivirus infection in a patient, the method comprising the step of administering to the patient a therapeutically effective amount of a fused protein or fragment thereof according to claim 1.

17. A fused protein according to claim 1 for use in the treatment or prevention of a flavivirus infection in a patient.

18. Use of a fused protein according to claim 1 in the preparation of a medicament for the treatment or prevention of a flavivirus infection in a patient.

19. A method for producing a fused protein, the method comprising:
(a). obtaining a gene clone derived from a flavivirus capsid protein;
(b). obtaining a biotin acceptor peptide gene;
(c). producing an expression vector through the joining of the gene clone derived from the flavivirus capsid protein and the biotin acceptor peptide gene;
(d). expressing the gene of the fused protein by transforming the expression vector to a host; and
(e). purifying the fused protein.

20. The method according to claim 19, wherein the host is Escherichia coli.

21. The method according to claim 19, wherein the purifying step includes the use of urea, non-ionic detergent, ion exchange chromatography and size exclusion chromatography.

22. (canceled)

23. A method of screening a library of molecules to identify or select one or more molecules thereof which selectively bind to a fused protein, or fragment thereof, according to claim 1, the method comprising:
(a) contacting the library of molecules with the fused protein; and
(b) detecting binding of one or more molecules to the fused protein.

24. The method according to claim 23, further comprising labelling the molecule with a biotin binding agent and binding is detected by detecting the labelled molecule.

25. The method according to claim 23, wherein the molecule is selected from a group consisting of antibodies and aptamers.

26. (canceled)
